(19) United States
(12) Patent Application Publication
(10) Pub. No.: US 2007/0218554 A1
(43) Pub. Date: Sep. 20, 2007

(54) Cell Culture Patterning Substrate And Method For Manufacturing The Same

(75) Inventors: Hideyuki Miyake, Tokyo (JP); Hideshi Hattori, Tokyo (JP); Hironori Kobayashi, Tokyo (JP)

Correspondence Address: LADAS & PARRY LLP, 224 SOUTH MICHIGAN AVENUE, SUITE 1600, CHICAGO, IL, 60604, US

(73) Assignee: DAI NIPPON PRINTING CO., LTD., Tokyo-To (JP)

(21) Appl. No.: 11/587,363

(22) PCT Filed: Apr. 25, 2005
(86) PCT No.: PCT/JP05/07775

§ 371 Date: Oct. 25, 2006

(30) Foreign Application Priority Data
Apr. 26, 2004 (JP) 2004-129721

(51) Int. Cl. C12N 5/02

(52) U.S. Cl. 435/395

(57) ABSTRACT

A main object of the present invention is to provide a cell culture patterning substrate and a method for manufacturing the same, which can be used to culture the cells on the substrate in an intended shape. To achieve the object, the present invention provides a cell culture patterning substrate comprising: a base material provided with a convex portion; and a cell culture region, which is a region for culturing a cell, formed on a surface of the base material, wherein the cell culture region is partitioned with the convex portion of the base material provided with the convex portion.

### TECHNICAL FIELD

The present invention relates to a method for manufacturing a cell culture patterning substrate capable of adhering cells in high-definition pattern.

### BACKGROUND ART

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culture dish with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method for arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in the patent document 1, an electric charge-retaining medium on which an electrostatic pattern is formed is applied to culture cells for the purpose of proliferating nerve cells in a form of circuit and the like. Furthermore, the patent document 2 tries to arrange cultured cells on a surface on which a cell adhesion-inhibiting or cell adhesive photosensitive hydrophilic polymer has been patterned by a photolithography method.

Furthermore, the patent document 3 discloses a cell culture base material on which a substance such as collagen and the like affecting on the adhesion ratio and form of cells is patterned, and a method for manufacturing this base material by a photolithography method. By culturing cells on such a base material, a larger amount of cells can be adhered on a surface on which collagen or the like is patterned, to realize patterning of cells.

However, even though the cells are patterned by the above-mentioned methods, there is a problem that the pattern is not maintained in some cases because the cells are bound to each other between the patterns.
Patent Document 1: Japanese Patent Application Laid-Open (JP-A) No. 2-245181
Patent Document 2: JP-A No. 3-7576
Patent Document 3: JP-A No. 5-176753

### DISCLOSURE OF INVENTION

### Problem to be Solved by the Invention

Accordingly, it has been desired to provide a cell culture patterning substrate and a method for manufacturing the same, which can be used to culture the cells on a substrate in an intended shape.

### Means for Solving the Problem

The present invention provides a cell culture patterning substrate comprising: a base material provided with a convex portion; and a cell culture region, which is a region for culturing a cell, formed on a surface of the base material, wherein the cell culture region is partitioned with the convex portion of the base material provided with the convex portion.

In the present invention, since the cell culture region is partitioned with the convex portion, bonding of the cells adhered to the adjacent cell culture regions can be prevented by the height, width, etc. of this convex portion so that the cell culture patterning substrate, capable of culturing cells in an intended pattern, can be obtained.

In the above-described invention, it is preferable that a region provided with the convex portion is a cell adhesion-inhibiting portion having adhesion-inhibiting properties to a cell, and the cell culture region is a cell adhesion portion having adhesive properties to a cell. Thereby, in the region provided with the convex portion, possibility of adhesion of cells can be made lower so that the cells can further be cultured in an objective pattern.

Such cell culture patterning substrate can be classified into the following six embodiments.

The first embodiment is a cell culture patterning substrate, wherein a photocatalyst-containing cell adhesion layer is formed on the base material, the photocatalyst-containing cell adhesion layer contains at least: a photocatalyst; and a cell adhesive material which has adhesive properties to a cell, and is to be decomposed or denatured by an action of a photocatalyst upon irradiation with energy, and in the cell adhesion-inhibiting portion, the cell adhesive material has been decomposed or denatured by an action of a photocatalyst upon irradiation with energy.

The second embodiment is a cell culture patterning substrate, wherein a photocatalyst-containing cell adhesion-inhibiting layer is formed on the base material, the photocatalyst-containing cell adhesion-inhibiting layer contains at least: a photocatalyst; and a cell adhesion-inhibiting material which has cell adhesion-inhibiting properties of inhibiting adhesion to a cell, and is to be decomposed or denatured by an action of a photocatalyst upon irradiation with energy, and in the cell adhesion portion, the cell adhesion-inhibiting material has been decomposed or denatured by an action of a photocatalyst upon irradiation with energy.

In the first and second embodiments, by irradiating the photocatalyst-containing cell adhesion layer or the photocatalyst-containing cell adhesion-inhibiting layer, containing a photocatalyst, with energy, the cell adhesive material or cell adhesion-inhibiting material is decomposed or denatured by an action of a photocatalyst contained in these layers themselves so that the cell adhesion portion and cell adhesion-inhibiting portion can be formed easily. Therefore, formation of the layer is easy because there is no need to form other separate layers. Thus, it is preferable from the point of view of manufacturing efficiency.

The third embodiment is a cell culture patterning substrate, wherein a photocatalyst-containing layer and a cell adhesion layer are formed on the base material, the photocatalyst-containing layer contains at least a photocatalyst, the cell adhesion layer contains a cell adhesive material which has adhesive properties to a cell, and is to be decomposed or denatured by an action of a photocatalyst upon irradiation with energy, and in the cell adhesion-inhibiting portion, the cell adhesive material has been decomposed or denatured by an action of a photocatalyst upon irradiation with energy.

The fourth embodiment is a cell culture patterning substrate, wherein a photocatalyst-containing layer and a cell adhesion-inhibiting layer are formed on the base material, the photocatalyst-containing layer contains at least a photocatalyst, the cell adhesion-inhibiting layer contains a cell adhesion-inhibiting material which has cell adhesion-inhibiting properties of inhibiting adhesion to a cell, and is to be decomposed or denatured by an action of a photocatalyst upon irradiation with energy, and in the cell adhesion portion, the cell adhesion-inhibiting material has been decomposed or denatured by an action of a photocatalyst upon irradiation with energy.

In the third and forth embodiments, by irradiating the photocatalyst-containing cell adhesion layer or the photocatalyst-containing cell adhesion-inhibiting layer, with energy, the photocatalyst contained in the adjacent photocatalyst-containing layer is excited so that the cell adhesive material or cell adhesion-inhibiting material can be decomposed or denatured. Thus, the cell adhesion portion and cell adhesion-inhibiting portion can be formed easily. Moreover, in these embodiments, since the possibility of the photocatalyst being exposed to the surface is low, when culturing the cells by adhering the cells on the cell culture region, there is also an advantage that the possibility of the cells being influenced by the photocatalyst with time is low.

The fifth embodiment is a cell culture patterning substrate, wherein a cell adhesion layer is formed, the cell adhesion layer contains at least a cell adhesive material which has cell adhesive properties of adhering to a cell, and is to be decomposed or denatured by an action of a photocatalyst upon irradiation with energy, and in the cell adhesion-inhibiting portion, the cell adhesive material has been decomposed or denatured by an action of a photocatalyst upon irradiation with energy.

The sixth embodiment is a cell culture patterning substrate, wherein a cell adhesion-inhibiting layer is formed on the base material, the cell adhesion-inhibiting layer contains at least a cell adhesion-inhibiting material which has cell adhesion-inhibiting properties of inhibiting adhesion to a cell, and is to be decomposed or denatured by an action of a photocatalyst upon irradiation with energy, and in the cell adhesion portion, the cell adhesion-inhibiting material has been decomposed or denatured by an action of a photocatalyst upon irradiation with energy.

In the fifth and sixth embodiments, by irradiating the cell adhesion layer or the cell adhesion-inhibiting layer with energy, for example by using the photocatalyst-containing layer side substrate or the like having the photocatalyst-containing layer containing at least a photocatalyst. Thus, the cell adhesive material or cell adhesion-inhibiting material can be decomposed or denatured easily by an action of a photocatalyst contained in the photocatalyst-containing layer so that the cell adhesion portion or cell adhesion-inhibiting portion can be formed. Moreover, in this case, since the photocatalyst is not needed to be contained in the cell adhesion layer or cell adhesion-inhibiting layer, the cells are not influenced by the photocatalyst with time. Therefore, there is an advantage that a high quality cell culture substrate can be obtained by adhering the cells.

The present invention provides a method for manufacturing a cell culture patterning substrate comprising energy irradiating process of forming a pattern containing a cell adhesion-inhibiting portion and a cell adhesion portion, wherein the cell adhesion-inhibiting portion is a portion whose cell adhesive material contained in a cell adhesion layer has been decomposed or denatured, the cell adhesion portion is a portion having adhesive properties to a cell and an energy is not irradiated thereto, a patterning substrate, comprising: a base material provided with a convex portion; and a cell adhesion layer containing a cell adhesive material which has adhesive properties to a cell and is to be decomposed or denatured by an action of a photocatalyst upon irradiation with energy, is used, a photocatalyst-containing layer side substrate, comprising a base body and a photocatalyst-containing layer containing a photocatalyst, is used, and the pattern is formed by disposing the patterning substrate and the photocatalyst-containing layer side substrate so that the cell adhesion layer and the photocatalyst-containing layer are facing to each other, and irradiating the region provided with the convex portion, with an energy, from a predetermined direction.

In the present invention, by irradiating the cell adhesion layer with energy by using the photocatalyst-containing layer side substrate, the cell adhesive material in the region provided with the convex portion is decomposed or denatured so that the region can be made into the cell adhesion-inhibiting portion having no adhesive properties to a cell. Therefore, when the cells are adhered to the cell adhesion portion, that is the cell culture region, of the cell culture patterning substrate manufactured in the present invention, bonding of the cells of the adjacent cell culture regions can be prevented due to the height and width of the convex portion provided on the base material, and the adhesion-inhibiting properties to a cell. Thus, the cell culture patterning substrate capable of culturing the cells in high-definition pattern can be obtained.

Moreover, the present invention provides a method for manufacturing a cell culture patterning substrate comprising energy irradiating process of forming a pattern containing a cell adhesion portion and a cell adhesion-inhibiting portion, wherein the cell adhesion portion is a portion whose cell adhesion-inhibiting material contained in a cell adhesion-inhibiting layer has been decomposed or denatured to have adhesive properties to a cell, the cell adhesion-inhibiting portion is a portion having cell adhesion-inhibiting properties of inhibiting adhesion to a cell and an energy is not irradiated thereto, a patterning substrate, comprising: a base material provided with a convex portion; and a cell adhesion-inhibiting layer containing a cell adhesion-inhibiting material which has cell adhesion-inhibiting properties of inhibiting adhesion to a cell and is to be decomposed or denatured by an action of a photocatalyst upon irradiation with energy, is used, a photocatalyst-containing layer side substrate, comprising a base body and a photocatalyst-containing layer containing a photocatalyst, is used, and the pattern is formed by disposing the patterning substrate and the photocatalyst-containing layer side substrate so that the cell adhesion layer and the photocatalyst-containing layer are facing to each other, and irradiating the region other than the region provided with the convex portion, with an energy, from a predetermined direction.

In the present invention, by irradiating the cell culture region on the cell adhesion-inhibiting layer with energy by using the photocatalyst-containing layer side substrate, the cell adhesion-inhibiting material is decomposed or denatured so that the region can be made into the cell adhesion portion having good adhesive properties to a cell. On the other hand, since the cell adhesion-inhibiting material remains in the region provided with the convex portion, which is the non-energy irradiated region, the region can be the cell adhesion-inhibiting portion. Therefore, when the cells are adhered and cultured on the cell adhesion portion, that is the cell culture region, of the cell culture patterning substrate manufactured in the present invention, bonding of the cells adhered to the adjacent cell culture regions can be prevented due to the height and width of the convex portion and to the adhesion-inhibiting properties of the cell adhesion-inhibiting layer. Thus, the cell culture patterning substrate capable of culturing the cells in high-definition pattern can be obtained.

### Effect of the Invention

According to the present invention, since the cell culture region is partitioned with the convex portion, bonding of the cells adhered to the adjacent cell culture regions can be prevented by the height of this convex portion so that the cell culture patterning substrate, capable of culturing cells in an intended pattern, can be obtained.

### BRIEF DESCRIPTION OF THE DRAWINGS

FIG. 1 is a schematic sectional view showing an example of the cell culture patterning substrate in the present invention.

FIGS. 2 are process drawings showing an example of a method for forming the cell adhesion portion and cell adhesion-inhibiting portion of the cell culture patterning substrate in the present invention.

FIG. 3 is a schematic sectional view showing an example of the photocatalyst-containing layer side substrate used in the present invention.

FIG. 4 is a schematic sectional view showing another example of the photocatalyst-containing layer side substrate used in the present invention.

FIG. 5 is a schematic sectional view showing another example of the photocatalyst-containing layer side substrate used in the present invention.

FIGS. 6 are illustrations showing an example of a method for forming the cell adhesion portion and cell adhesion-inhibiting portion of the cell culture patterning substrate in the present invention.

FIGS. 7 are process drawings showing an example of a method for forming the cell culture patterning substrate in the present invention.

### DESCRIPTION OF SYMBOLS

1: Base material
2: Convex portion
3: Cell culture region
4: Photocatalyst-containing cell adhesion layer
6: Energy
7: Cell adhesion-inhibiting portion
8: Cell adhesion layer
9: Cell adhesion portion
11: Base body
12: Photocatalyst-containing layer
13: Photocatalyst-containing layer side substrate

### BEST MODE FOR CARRYING OUT THE INVENTION

The present invention relates to a cell culture patterning substrate used for adhering and culturing cells on a base material in a high-definition pattern, and to a method for manufacturing the same. Hereinafter, each will be explained separately.

A. Cell Culture Patterning Substrate

First, the cell culture patterning substrate of the present invention will be explained. The cell culture patterning substrate of the present invention comprises: a base material provided with a convex portion; and a cell culture region, which is a region for culturing a cell, formed on a surface of the base material, wherein the cell culture region is partitioned with the convex portion of the base material provided with the convex portion.

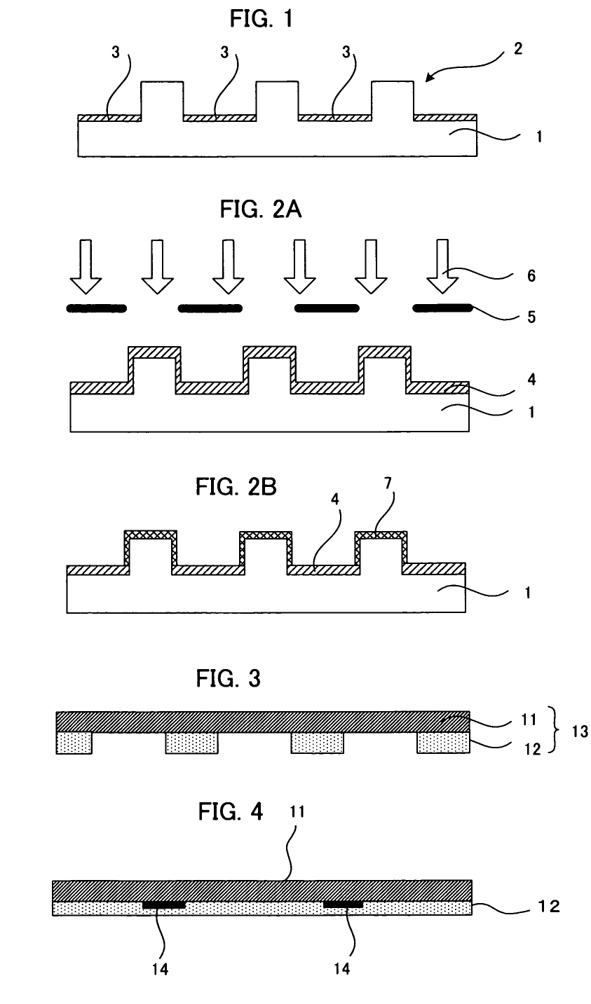

For example as shown in FIG. 1, in the cell culture patterning substrate of the present invention, the cell culture region 3, which is the region for culturing the cells, is partitioned with the convex portion 2 provided on the base material 1.

In the present invention, since the above-mentioned convex portion is formed in between the adjacent cell culture regions, adhesion of the cells adhered on the adjacent cell culture regions can be prevented by the height, the width, etc. of the convex portion. Thereby, a cell culture patterning substrate capable of culturing the cells in an intended pattern can be provided. Hereinafter, each configuration of the cell culture patterning substrate of the present invention will be explained.

(Base Material)

First, the base material, provided with the convex portion, used in the present invention will be explained. The base material, provided with the convex portion, used in the present invention is not particularly limited as long as the bonding of the cells adhered on the adjacent cell culture regions, to each other, can be prevented by the convex portion. For example, an inorganic material such as metal, glass and silicon, an organic material represented by a plastic, or the like can be used.

Moreover, the flexibility, the transparency, etc. of the base material can be selected optionally according to the kind, the application, etc. of the cell culture patterning substrate.

Moreover, as the method for forming the convex portion, a common method for forming a base material can be used. For example, a method of molding a material into a shape having a convex portion, a method of attaching a member having a shape of a convex portion onto a flat member, a method of printing a convex portion onto a flat member, a method of providing a convex portion by coating a photo resist on a flat member by a photolithography method, a method of providing a convex portion by cutting a flat member, a method of providing a convex portion by providing a coating layer on a flat member and etching the coating layer, a method of molding a resin by a thermal press using a mold having a concavoconvex shape, a method of forming by pouring a curable resin into a mold and curing, or the like can be presented.

In the case of using the above-mentioned method of attaching a member having a convex portion on a flat member, the flat member and the member to be attached may be of different materials, or of the same member. For example, the flat member having cell adhesive properties may be used, and a material having no adhesive properties to a cell may be used for the member to be attached as the convex portion. Thereby, the region not provided with the convex portion can be the cell culture region having the adhesive properties to a cell, and the region provided with the convex portion can be the cell adhesion-inhibiting region having no adhesive properties to a cell. Moreover, also in the case of using a method of printing a convex portion on a flat member, the convex portion may be printed using the same material as the material of the flat member, or it may be printed, using a material having no adhesive properties to a cell, on a flat member having the cell adhesive properties.

Moreover, the shape of the above-mentioned convex portion is not particularly limited as long as it can inhibit bonding of the cells adhered on the adjacent cell culture regions. For example, the cross-sectional shape may be rectangular, circular, semi-circular, etc. Furthermore, it may be triangular, or the like.

Here, the height of the above-mentioned convex portion can be selected optionally according to the size of the cells to be cultured, or the like, and it is generally about 0.1 μm to 1,000 μm, more preferably about 1 μm to 500 μm, and particularly preferably about 10 μm to 200 μm. Moreover, the width of the convex portion is generally about 10 μm to 1,000 μm, more preferably about 50 μm to 800 μm, and particularly preferably about 100 μm to 500 μm. Here, the above-mentioned width refers to the width of the bottom portion of the formed convex portion. By providing the convex portion having such width and height, bonding of the cells adhered on the adjacent cell culture regions can be prevented. The width of the above-mentioned convex portion may either be wider or narrower than the width between the adjacent convex portions, that is, the width of the cell culture region.

(Cell Culture Region)

Next, the cell culture region in the cell culture patterning substrate of the present invention will be explained. The cell culture region in the present invention is a region used for culturing the cells, and also a region partitioned with the above-mentioned convex portion so that the shape of the cell culture region can be selected optionally according to the kind of the cells, the purpose, or the like. Here, “partitioned with the convex portion” means that the above-mentioned convex portion may be formed on the entire surface of the circumference of the cell culture region. Alternatively, the convex portion may be formed only in a part of the circumference of the cell culture region, thus partitioning the cell culture region.

In the present invention, the above-mentioned cell culture region is not particularly limited as long as it is formed to have preferable adhesive properties to a cell. For example, the adhesive properties to a cell may be imparted by biochemical characteristics, or may the adhesive properties to a cell may be imparted by physiochemical properties, of the like. Moreover, in the case the adhesive properties to a cell differ depending on the kind of the cells, or the like, one capable of preferably adhering to the object cells can be used.

Such a cell culture region, for example, may have a cell adhesive layer formed on a base material using a cell adhesive material having the adhesive properties to a cell. Alternatively, as described above, the cell culture region may be obtained by using one having the adhesive properties to a cell as the base material.

For example, as a material having the adhesive properties to a cell that can also be used as a base material, a silicone resin, a styrene resin, an acrylic resin, a methacrylic resin, a polyester, a polyethylene, a polyglycolic acid, a polylactic acid, a quartz glass, a soda glass, apatites, aluminas, or the like can be presented. Moreover, as the cell adhesive material used for the above-mentioned cell adhesive layer, a cell adhesive material used for a common cell culture substrate or the like can be used. For example, as the material which adheres to a cell due to the physiochemical properties, for example, hydrophilic polystyrene, poly(N-isopropyl acrylamide), basic polymers such as polylysine, basic compounds such as amino propyl triethoxysilane, N-(2-aminoethyl)-3-aminopropyltrimethoxysilane etc., and condensates containing thereof can be listed. Moreover, the cell adhesive material having adhesive properties to a cell biochemically includes fibronectin, laminine, tenascin, vitronectin, an RGD (arginine-glycine-aspartic acid) sequence-containing peptide, a YIGSR (tyrosine-isoleucine-glycine-serine-arginine) sequence-containing peptide, collagen, atelocollagen, gelatin, etc.

(Cell Culture Patterning Substrate)

The cell culture patterning substrate of the present invention is not particularly limited as long as it has the above-mentioned cell culture region on the base material mentioned above. In the present invention, it is particularly preferable that the region provided with the convex portion is a cell adhesion-inhibiting portion having the cell adhesion-inhibiting properties of inhibiting adhesion to a cell, and the cell culture region for culturing the cells is a cell adhesion portion having adhesive properties to a cell. Thereby, not only by the height and the width of the above-mentioned convex portion, but also by the cell adhesion-inhibiting properties of the convex portion, adhesion of the cells in the adjacent cell culture regions can be prevented so that formation of the cells in an intended pattern can further be facilitated. “The region provided with the convex portion has the cell adhesion-inhibiting properties” includes not only the case wherein the above-mentioned convex portion itself has the cell adhesion-inhibiting properties to a cell, but also the case wherein the cell adhesion-inhibiting properties are imparted by forming a layer having the cell adhesion-inhibiting properties, on the convex portion.

Examples of the method for forming the cell culture patterning substrate having such cell adhesion portion and cell adhesion-inhibiting portion are as follows: it may be formed by forming the cell adhesion layer, having adhesive properties to a cell, on the base material comprising the material having cell adhesion-inhibiting properties so that the convex portion has the cell adhesion-inhibiting properties, and the cell adhesion region has the cell adhesive properties; it may be formed by forming the cell adhesion-inhibiting layer, having the cell adhesion-inhibiting properties, on the convex portion of the base material having the cell adhesive properties; and it may be formed by forming the cell adhesion layer in the cell culture region of the base material provided with the convex portion and the cell adhesion-inhibiting layer on the convex portion.

Here, in the present invention, it is particularly preferable that the above-mentioned cell adhesion portion and cell adhesion-inhibiting portion are formed by using a layer whose adhesive properties to a cell is varied by an action of a photocatalyst upon irradiation with energy. Thereby, patterning of the cell adhesion portion and the cell adhesion-inhibiting portion can be facilitated.

Hereafter, the cell adhesion portion and cell adhesion-inhibiting portion formed by an action of a photocatalyst upon irradiation with energy will be explained. As the embodiments thereof, following six embodiments can be presented. Each embodiment will be explained in detail.

### (1) FIRST EMBODIMENT

First, the first embodiment is the cell culture patterning substrate, wherein a photocatalyst-containing cell adhesion layer is formed on the base material provided with a convex portion, the photocatalyst-containing cell adhesion layer contains at least: a photocatalyst; and a cell adhesive material which has adhesive properties to a cell, and is to be decomposed or denatured by an action of a photocatalyst upon irradiation with energy, and in the cell adhesion-inhibiting portion, the cell adhesive material has been decomposed or denatured by an action of a photocatalyst upon irradiation with energy.

In this embodiment, since the photocatalyst-containing cell adhesion layer contains the photocatalyst and the cell adhesive material, a cell adhesion-inhibiting portion, to which the cells do not adhere, can be provided by decomposing or denaturing the cell adhesive material by an action of the photocatalyst by irradiating the energy to the photocatalyst-containing cell adhesion layer on the region provided with a convex portion. On the other hand, since the cell adhesive material remains in the region without the energy irradiation, a cell adhesion portion having preferable adhesive properties to a cell can be provided. Therefore, without the need of a special device, a complicated process or the like, by irradiating the energy in a pattern, the cell adhesion-inhibiting portion, which has no adhesive properties to a cell, can be formed easily in the cell culture region that is the cell adhesion portion.

Moreover, in this embodiment, when the cell culture substrate is obtained by adhering the cells onto the cell culture region of the cell culture patterning substrate, the cells adhered to the cell adhesion-inhibiting portion can be removed due to an action of a photocatalyst, by irradiating energy only to the cell adhesion-inhibiting portion provided with the convex portion. Thereby, there is an advantage that the cells cultured in high-definition pattern can be maintained.

Hereinafter, the photocatalyst-containing cell adhesion layer and the base material used in this embodiment will be explained. Furthermore, the method for forming the cell adhesion-inhibiting portion will be explained.

a. Photocatalyst-Containing Cell Adhesion Layer

First, the photocatalyst-containing cell adhesion layer used in this embodiment will be explained. The photocatalyst-containing cell adhesion layer used in this embodiment contains at least a photocatalyst and the cell adhesive material. Also, this layer will be a layer having low adhesive properties to a cell by decomposing or denaturing the cell adhesive material by an action of a photocatalyst upon irradiation with energy.

The photocatalyst-containing cell adhesion layer can be formed by coating, etc. a photocatalyst-containing cell adhesion layer forming coating solution, containing a photocatalyst and a cell adhesive material to be decomposed or denatured by an action of a photocatalyst upon irradiation with energy, onto a base material provided with a convex portion. The photocatalyst-containing cell adhesion layer forming coating solution can be coated by a common coating method. For example, the spin coating method, the spray coating method, the dip coating method, the roll coating method, the bead coating method or the like can be used.

At the time, the film thickness of the photocatalyst-containing cell adhesion layer can be selected optionally according to the kind, etc. of the cell culture patterning substrate. It is in general about 0.01 μm to 1.0 μm, in particular, about 0.1 μm to 0.3 μm.

Hereinafter, each material used for the photocatalyst-containing cell adhesion layer used in this embodiment will be explained.

(i) Cell Adhesive Material

First, the cell adhesive material contained in the photocatalyst-containing cell adhesion layer of this embodiment will be explained. The kind, etc. of the cell adhesive material contained in the photocatalyst-containing cell adhesion layer is not particularly limited as long as it has the adhesive properties to a cell and it is decomposed or denatured by an action of a photocatalyst upon irradiation with energy. Here, “having adhesive properties to a cell” means being good in adhesion to a cell. For instance, when the adhesive properties to a cell differ depending on the kind of cells, it means to be good in the adhesion with target cells.

The cell adhesive material used in the present embodiment has such adhesive properties to a cell. Those losing adhesive properties to a cell, or obtaining the cell adhesion-inhibiting properties of inhibiting adhesion to a cell, by being decomposed or denatured by an action of a photocatalyst upon irradiation with energy, are used.

As such materials having the adhesive properties to a cell, there are two kinds. One being material having the adhesive properties to a cell owing to physicochemical characteristics and the other being material having the adhesive properties to a cell owing to biochemical characteristics.

As physicochemical factors that determine the adhesive properties to a cell of the materials having the adhesive properties to a cell owing to the physicochemical characteristics, the surface free energy, the electrostatic interaction and the like can be cited. For instance, when the adhesive properties to a cell is determined by the surface free energy of the material, if the material has the surface free energy in a predetermined range, the adhesive properties between the cells and the material becomes good. If it deviates from the predetermined range, the adhesive properties between the cells and the material will be lower. As such changes of the adhesive properties to a cell due to the surface free energy, experimental results shown in Data, for instance, CMC Publishing Co., Ltd. “Biomaterial no Saisentan”, Yoshito Ikada (editor), p. 109, lower part are known. As materials having the adhesive properties to a cell owing to such a factor, for instance, hydrophilic polystyrene, poly (N-isopropyl acrylamide) and the like can be cited. When such a material is used, by the action of a photocatalyst upon irradiation with energy, for instance, a functional group on a surface of the material is substituted, decomposed or the like to cause a change in the surface free energy, resulting in one having no adhesive properties to a cell, or one having the cell adhesion-inhibiting properties.

When the adhesive properties between cells and a material is determined owing to the electrostatic interaction or the like, for instance, the adhesive properties to a cell are determined by an amount of positive electric charges and the like that the material has. As materials having the adhesive properties to a cell owing to such electrostatic interaction, basic polymers such as polylysine; basic compounds such as aminopropyltriethoxysilane, N-(2-aminoethyl)-3-aminopropyltrimethoxysilane; and condensates and the like including these can be cited. When such materials are used, by the action of a photocatalyst upon irradiation with energy, the above-mentioned materials are decomposed or denatured. Thereby, for instance, an amount of positive electric charges present on a surface can be altered, resulting in one having no adhesive properties to a cell, or one having the cell adhesion-inhibiting properties.

As materials having the adhesive properties to a cell owing to the biological characteristics, ones that are good in the adhesive properties with particular cells or ones that are good in the adhesive properties with many cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like, can be cited. When such material is used, by the action of a photocatalyst upon irradiation with energy, for instance, a structure of the material is partially destroyed, or a principal chain is destroyed or the like, resulting in one having no adhesive properties to a cell, or one having the cell adhesion-inhibiting properties can be obtained.

Such a cell adhesive material, though it differs depending on the kind of the materials and the like, is comprised in the photocatalyst-containing cell adhesion layer normally in the range of 0.01% by weight to 95% by weight, and preferably in the range of 1% by weight to 10% by weight. Thereby, a region that contains the cell adhesive material can be made a region good in the adhesive properties to a cell.

(ii) Photocatalyst

Next, the photocatalyst contained in the photocatalyst-containing cell adhesion layer of this embodiment will be explained. The photocatalyst used in this embodiment is not particularly limited as long as it can decompose or denature the above-mentioned cell adhesive material by an action of a photocatalyst upon irradiation with energy.

Here, although the function mechanism of the photocatalyst represented by a titanium oxide to be described later is not always clear, it is considered that the carrier produced by the light irradiation changes the chemical structure of an organic substance by the direct reaction with a compound in the vicinity or by the active oxygen species generated in the presence of oxygen and water. In this embodiment, the carrier is considered to act on the cell adhesive material.

As the photocatalyst that can be used in the present embodiment, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present embodiment, in particular, titanium dioxide can be preferably used owing to a large band gap, chemical stability, non-toxicity, and easy availability. There are two types of titanium dioxide, anatase type and rutile type, and both can be used in the present embodiment; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-KO1, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. It is preferable to use the photocatalyst with an average particle diameter of 50 nm or less, and one having an average particle diameter of 20 nm or less can be particularly preferably used.

The content of the photocatalyst in the photocatalyst-containing cell adhesion layer in this embodiment can be set in the range of 5 to 95% by weight, preferably 10 to 60% by weight, and further preferably 20 to 40% by weight.

Thereby, the cell adhesive material in the energy-irradiated region of the photocatalyst-containing cell adhesion layer can be decomposed or denatured.

Here, it is preferable that the photocatalyst used in this embodiment has low adhesive properties to a cell by, for example, having high hydrophilic properties or the like. Thereby, the region, whose cell adhesive material is decomposed or the like to expose the photocatalyst, can be used as a region having low adhesive properties to a cell.

(iii) Others

In this embodiment, not only the cell adhesive material and photocatalyst, but also a binder etc. for improving strength, resistance etc. may be contained as necessity in the photocatalyst-containing cell adhesion layer. In the present embodiment, particularly as the binder, at least after the energy irradiation, a material having the cell adhesion-inhibiting properties of inhibiting adhesion to cells is preferably used. This is because the cell adhesive properties of the cell adhesion-inhibiting portion, which is a region irradiated with energy, can thereby be made lower. As such a material, one having the cell adhesion-inhibiting properties prior to the energy irradiation or one obtaining the cell adhesion-inhibiting properties by the action of a photocatalyst upon irradiation with energy may be used.

In the present embodiment, a material that becomes to have the cell adhesion-inhibiting properties, particularly by the action of a photocatalyst upon irradiation with energy, is preferably used as a binder. Thereby, in a region prior to the energy irradiation, the adhesion between the cell adhesive material and cells is not inhibited, and only a region where energy is irradiated can be lowered in the adhesive properties to a cell.

As materials that can be used as such a binder, for instance, ones whose main skeleton has such a high bond energy that cannot be decomposed by the photo-excitation of the photocatalyst, and having an organic substituent which can be decomposed by the action of the photocatalyst are preferably used. For instance, (1) organopolysiloxane that exhibits large strength by hydrolyzing or polycondensating chloro- or alkoxysilane or the like by a sol-gel reaction and the like, and (2) organopolysiloxane and the like in which reactive silicones excellent in the water repellency or oil repellency are crosslinked, can be cited.

In the case of the (1), it is preferable to be organopolysiloxanes that are hydrolysis condensates or cohydrolysis condensates of one kind or two or more kinds of silicon compounds expressed by a general formula:
YₙSiX₍₄₋ₙ₎
(Here, Y denotes an alkyl group, fluoroalkyl group, vinyl group, amino group, phenyl group, epoxy group or organic group containing the above, and X denotes an alkoxyl group, acetyl group or halogen. “n” is an integer of 0 to 3.). The number of carbons of the organic group expressed with Y is preferably in the range of 1 to 20, and the alkoxy group expressed with X is preferably a methoxy group, ethoxy group, propoxy group or butoxy group.

As the reactive silicone according to the (2) compounds having a skeleton expressed by a general formula below can be cited.

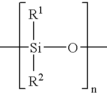

In the above general formula, n denotes an integer of 2 or more, R¹ and R² each represents a substituted or nonsubstituted alkyl group, alkenyl group, aryl group or cyanoalkyl group having 1 to 20 carbons, and a vinyl, phenyl and halogenated phenyl occupy 40% or less by mole ratio to a total mole. Furthermore, one in which R¹ and R² is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain. When the material mentioned above is used, by the action of a photocatalyst upon irradiation with energy, a surface of an energy-irradiated region can be made high in the hydrophilicity. Thereby, the adhesion to cells is inhibited, and the region where energy is irradiated can be made into a region on which the cells do not adhere.

Together with the above-mentioned organopolysiloxanes, a stable organo silicium compound that does not cause a crosslinking reaction, such as dimethylpolysiloxanes, may be blended with a binder.

When the above-mentioned material is used as the cell adhesion-inhibiting material, the contact angle thereof with water is preferably in the range of 15° to 120°, more preferably 20° to 100° before the material is irradiated with energy. According to this, the adhesion of the cell adhesive material to cells is not inhibited.

In the case of irradiating this cell adhesion-inhibiting material with energy, it is preferred that the contact angle thereof with water becomes 10° or less. This range makes it possible to render the material having a high hydrophilicity and low adhesive properties to a cell.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

In the present embodiment, a decomposition substance or the like that causes such as a change in the wettability of a region where energy is irradiated, thereby lowers the adhesive properties to a cell or that aides such a change, may be contained.

As such decomposition substances, for instance, surfactants or the like that are decomposed and the like, by the action of a photocatalyst upon irradiation with energy, to be hydrophilic and the like to result in lowering the adhesive properties to a cell can be cited. Specifically, nonionic surfactants: hydrocarbon based such as respective series of NIKKOL BL, BC, BO, and BB manufactured by Nikko Chemicals Co., Ltd.; and silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by Neos, UNIDYNE DS-401 and 402 manufactured by DAIKIN INDUSTRIES, Ltd., and Fluorad FC-170 and 176 manufactured by 3M can be cited. Cationic surfactants, anionic surfactants and amphoteric surfactants also can be used.

Other than the surfactants, oligomers and polymers such as polyvinyl alcohol, unsaturated polyester, acrylic resin, polyethylene, diallyl phthalate, ethylene propylene diene monomer, epoxy resin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, polyisoprene and the like can be cited.

In the present embodiment, such a binder can be preferably contained in the photocatalyst-containing cell adhesion layer, in the range of 5% by weight to 95% by weight, more preferably 40% by weight to 90% by weight, and particularly preferably 60% by weight to 80% by weight.

b. Base Material

Next, the base material used in the present embodiment will be explained. The base material used in the present embodiment is not particularly limited as long as it is provided with the above-described convex portion. As such base material, for example, an inorganic material such as metal, glass and silicon, or an organic material typified by plastics and the like can be used.

Moreover, the flexibility or the like of the base material can be selected optionally according to the kind, the application or the like of the cell culture patterning substrate. Moreover, the transparency of the base material can be selected optionally according to the kind of the cell culture patterning substrate or the irradiation direction of the energy to be irradiated for decomposing or denaturing the cell adhesive material or the like. For example, in the case the base material has the light-shielding portion or the like, and the energy irradiation is carried out from the base material side or the like, the base material needs to have the transparency.

The method for forming the above-described convex portion is not particularly limited as long as it is a method capable of forming the above-described convex portion. Since the method can be a common method used to form a base material, and the method can be the same as the above-described method, detailed explanation is omitted.

Here, in this embodiment, when the base material has transparency to the irradiated energy, in order to prevent the cell culture region to be irradiated with the energy, a light-shielding portion may be formed in between the base material and the photocatalyst-containing cell adhesion layer, the position corresponding to the cell culture region. Thereby, at the time of forming the cell adhesion-inhibiting portion by irradiating the energy to the region provided with a convex portion, by irradiating the energy on the entire surface from the rear side of the base material, without the use of a photomask or the like, the cell adhesive material in the photocatalyst-containing cell adhesion layer formed on the convex portion can be decomposed or denatured.

The light-shielding portion to be used in this embodiment is not particularly limited as long as it can shield the energy to be irradiated to the cell culture patterning substrate at the time of forming the cell adhesion-inhibiting portion. For example, it may be formed by forming a thin film of a metal such as chromium, in about a 1,000 to 2,000 Å thickness, by a sputtering method, a vacuum deposition method or the like, and then, patterning the thin film. As the patterning method, an ordinary patterning method such as sputtering can be used.

Moreover, it may be formed by a method in which a layer containing light-shielding particles such as carbon particulates, metal oxides, inorganic pigments and organic pigments in a resin binder is formed in a pattern. As the resin binders that can be used, a polyimide resin, acrylic resin, epoxy resin, polyacrylamide, polyvinyl alcohol, gelatin, casein, cellulose and the like can be used singularly or in combination of two or more kinds. Furthermore, a photosensitive resin and an O/W emulsion type resin composition such as emulsified reactive silicone can be used. A thickness of such the resinous light-shielding portion can be set in the range of 0.5 to 10 μm. As a method for patterning such the resinous light-shielding portion, methods such as a photolithography method and a printing method that are generally used can be used.

c. Method for Forming a Cell Adhesion-Inhibiting Portion

Next, the method for forming a cell adhesion-inhibiting portion in this embodiment will be explained. In this embodiment, for example as shown in FIG. 2, by irradiating the energy 6 in a pattern of a cell adhesion-inhibiting portion to be formed, that is the region provided with the convex portion, by using for example a photomask 5 or the like, to the photocatalyst-containing cell adhesion layer 4 formed on the base material 1 provided with the convex portion (FIG. 2A), the cell adhesive material in the photocatalyst-containing cell adhesion layer 4, of the region irradiated with the energy, is decomposed or denatured. Thus, a cell adhesion-inhibiting portion 7 having no adhesive properties to a cell can be formed (FIG. 2B). At the time, the cell adhesion-inhibiting portion contains the photocatalyst and the decomposed product or the denatured product of the cell adhesive material.

The energy irradiation (exposure) mentioned in this embodiment is a concept that includes all energy ray irradiation that can decompose or denature the cell adhesive material by the action of a photocatalyst upon irradiation with energy, and is not limited to light irradiation.

Normally, the wavelength of the light used in such energy irradiation is set in the range of 400 nm or less, preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which pattern irradiation is carried out via a photomask by using the above-mentioned light source, a method of carrying out drawing irradiation in a pattern by using laser such as excimer, YAG and the like can be used. Furthermore, as mentioned above, when the base material has the light-shielding portion in a pattern same as that of the cell adhesion portion, energy can be irradiated over the entire surface from the base material side. In this case, there are advantages in that there are no needs of the photomask and the like and a process of positional alignment and the like are also not necessary.

An amount of irradiation of energy at the energy irradiation is an amount of irradiation necessary for decomposing or denaturing the cell adhesive material by the action of the photocatalyst.

At this time, by irradiating a layer containing the photocatalyst, with energy, while heating, the sensitivity can be raised; accordingly, it is preferable in that the cell adhesive material can be efficiently decomposed or denatured. Specifically, it is preferable to heat in the range of 30° C. to 80° C.

The energy irradiation that is carried out via a photomask in this embodiment, when the above-mentioned base material is transparent, may be carried out from either direction of the base material side or a photocatalyst-containing cell adhesion layer side. On the other hand, when the base material is opaque, it is necessary to irradiate energy from a photocatalyst-containing cell adhesion layer side.

### (2) SECOND EMBODIMENT

First, the second embodiment is the cell culture patterning substrate, wherein a photocatalyst-containing cell adhesion-inhibiting layer is formed on the base material provided with the convex portion, the photocatalyst-containing cell adhesion-inhibiting layer contains at least: a photocatalyst; and a cell adhesion-inhibiting material which has cell adhesion-inhibiting properties of inhibiting adhesion to a cell, and is to be decomposed or denatured by an action of a photocatalyst upon irradiation with energy, and in the cell adhesion portion, the cell adhesion-inhibiting material has been decomposed or denatured by an action of a photocatalyst upon irradiation with energy.

In this embodiment, since the photocatalyst-containing cell adhesion-inhibiting layer contains the photocatalyst and the cell adhesion-inhibiting material, by irradiating the energy to the region other than the convex portion of the photocatalyst-containing cell adhesion-inhibiting layer, the cell adhesion-inhibiting material can be decomposed or denatured by an action of the photocatalyst contained in the layer. Thus, the energy-irradiated region can be made into the cell adhesion portion having adhesive properties to a cell, that is, the cell culture region. At the same time, in the region provided with the convex portion, which is not irradiated with energy, the cell adhesion-inhibiting material still remains so that the region has the cell adhesion-inhibiting properties.

Hereinafter, the photocatalyst-containing cell adhesion-inhibiting layer and the base material used in this embodiment will be explained. Furthermore, the method for forming the cell adhesion portion will be explained.

a. Photocatalyst-Containing Cell Adhesion-Inhibiting Layer

First, the photocatalyst-containing cell adhesion-inhibiting layer used in this embodiment will be explained. The photocatalyst-containing cell adhesion-inhibiting layer used in this embodiment contains a photocatalyst and the cell adhesion-inhibiting material. Also, this layer will be a layer having the adhesive properties to a cell when the cell adhesion-inhibiting material is decomposed or denatured by an action of a photocatalyst upon irradiation with energy.

The photocatalyst-containing cell adhesion-inhibiting layer can be formed by coating, etc. a photocatalyst-containing cell adhesion-inhibiting layer forming coating solution, containing a photocatalyst and a cell adhesive material to be decomposed or denatured by an action of a photocatalyst upon irradiation with energy, onto a base material provided with the convex portion. The photocatalyst-containing cell adhesion-inhibiting layer forming coating solution can be coated by a common coating method. For example, the spin coating method, the spray coating method, the dip coating method, the roll coating method, the bead coating method or the like can be used.

At the time, the film thickness of the photocatalyst-containing cell adhesion-inhibiting layer can be selected optionally according to the kind, etc. of the cell culture patterning substrate. It is in general about 0.01 μm to 1.0 μm, in particular, about 0.1 μm to 0.3 μm.

Hereinafter, the materials used for the photocatalyst-containing cell adhesion-inhibiting layer used in this embodiment will be explained. Since the photocatalyst used in this embodiment may be the same as the photocatalyst used in the above-described first embodiment. Therefore, detailed description is omitted.

(i) Cell Adhesion-Inhibiting Material

First, the cell adhesion-inhibiting material contained in the photocatalyst-containing cell adhesion-inhibiting layer used in this embodiment will be explained.

The kind or the like of the cell adhesion-inhibiting material used in this embodiment is not particularly limited as long as it has the cell-adhesion inhibiting properties of inhibiting adhesion to a cell, and also, it is decomposed or denatured by an action of a photocatalyst upon the irradiation with energy.

Here, “to have the cell adhesion-inhibiting properties” refers to a nature of inhibiting the cells from adhering to the cell adhesion-inhibiting material. In the case the adhesive properties to a cell differ depending upon the kind of the cells, it means to have the nature inhibiting the adhesion to the object cells.

As the cell adhesion-inhibiting material used in this embodiment, those having such cell adhesion-inhibiting properties that are decomposed or denatured by an action of a photocatalyst upon irradiation with energy so as to lose the cell adhesion-inhibiting properties, or so as to obtain preferable adhesive properties to a cell, can be used.

As the cell adhesion-inhibiting material, for example, a material having high hydration ability can be used. The material having high hydration ability has a hydration layer, wherein water molecules gather around. In general, since adhesive properties to the water molecules of such a substance is higher than the adhesive properties to a cell, the cells cannot be adhered to the above-mentioned material having high hydration ability so as to have low adhesive properties to a cell. “Hydration ability” refers to a nature to be hydrated with the water molecules, and “to have high hydration ability” means to be easily hydrated with the water molecules.

As the material having high hydration ability so as to be used as the cell adhesion-inhibiting material, for example, a polyethylene glycol, an amphoteric ion material having a betaine structure, a phosphoric lipid, or the like can be presented. In a case such material is used as the above-mentioned cell adhesion-inhibiting material, at the time of irradiating energy in the below-described energy irradiation process, the cell adhesion-inhibiting material is decomposed or denatured by an action of a photocatalyst. Thus, due to the separation of the hydration layer on the surface, one having no cell adhesion-inhibiting properties can be provided.

Moreover, in this embodiment, as the above-mentioned cell adhesion-inhibiting material, a surfactant having water repellent or oil repellent organic substituent to be decomposed by an action of a photocatalyst can also be used. As such a surfactant, for example, nonionic surfactants: hydrocarbon based such as respective series of NIKKOL BL, BC, BO, and BB manufactured by Nikko Chemicals Co., Ltd.; and fluorine based or silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by Neos, UNIDYNE DS-401 and 402 manufactured by DAIKIN INDUSTRIES, Ltd., and Fluorad FC-170 and 176 manufactured by 3M can be cited. Cationic surfactants, anionic surfactants and amphoteric surfactants also can be used.

At the time of forming the photocatalyst-containing cell adhesion-inhibiting layer using such a material as the cell adhesion-inhibiting material, the cell adhesion-inhibiting material is distributed unevenly on the surface. Thereby, the water repellency and the oil repellency of the surface can be made higher so as to provide one having small interaction with the cells and the low adhesive properties to a cell can be provided. Moreover, when the energy is irradiated to the layer in the energy irradiation process, it is easily decomposed by an action of a photocatalyst so as to expose the above-mentioned photocatalyst. Thus, one not having the above-mentioned cell adhesion-inhibiting properties can be obtained.

In this embodiment, as the cell adhesion-inhibiting material, it is particularly preferable to use one obtains good adhesive properties to a cell by an action of a photocatalyst upon irradiation with energy. As such a cell adhesion-inhibiting material, for example, materials having the oil repellency or the water repellency can be presented.

In the case the material having the oil repellency or the water repellency is used as the cell adhesion-inhibiting material, owing to the water repellency or the oil repellency of the cell adhesion-inhibiting material, the interaction, such as the hydrophobic interaction, between the cells and the cell adhesion-inhibiting material can be made low so that the adhesive properties to a cell can also be lowed.

As such a material having the water repellency or the oil repellency, for example one having a skeleton with high bonding energy so as not to be decomposed by an action of a photocatalyst which having a water repellent or oil repellent organic substituent to be decomposed by an action of a photocatalyst, or the like can be presented.

As the one having a skeleton with high bonding energy so as not to be decomposed by an action of a photocatalyst which having a water repellent or oil repellent organic substituent to be decomposed by an action of a photocatalyst, for example, (1) organopolysiloxane that exhibits large strength by hydrolyzing or polycondensating chloro- or alkoxysilane or the like by a sol-gel reaction and the like, and (2) organopolysiloxane and the like in which reactive silicones are crosslinked, that are used as a binder in the above-described first embodiment can be presented.

In the case of being used as a binder in the first embodiment, such a substance can be used as a material having the cell adhesion-inhibiting properties by providing ultra hydrophilic properties by decomposing or denaturing the side chain of the above-mentioned organopolysiloxane, at high ratio, by an action of a photocatalyst upon irradiation with energy. However, in this embodiment, the energy-irradiated region can be imparted with the adhesive properties to a cell by irradiating with the energy to the extent so as the side chain of the organopolysiloxane is not to completely decomposed or denatured by an action of a photocatalyst upon irradiation with energy. Moreover, together with the organopolysiloxane, a stable organosilicone compound that is not cross-linked, such as a dimethyl polysiloxane, may be mixed additionally.

In the case of using the material having the water repellency or the oil repellency as the cell adhesion-inhibiting material, in general, it is preferable to use a material having the contact angle with water of 80° or more, and more preferably in the range of 100° to 130° as the cell adhesion-inhibiting material. Thereby, the photocatalyst-containing cell adhesion-inhibiting layer before the energy irradiation can have low adhesive properties to a cell. The upper limit of the above-mentioned angle is the upper limit of the contact angle with water of the cell adhesion-inhibiting material on a flat base material. For example, in the case of measuring the contact angle with water of the cell adhesion-inhibiting material on a base material having the concavoconvex, the upper limit may be about 160° as it is shown in the material Japanese Journal of Applied Physics, part 2, vol. 32, L614 to L615, 1993, by Ogawa, et al.

Moreover, in the case of providing the adhesive properties to a cell by irradiating the energy to the cell adhesion-inhibiting material, it is preferable to irradiate the energy so as to have the contact angle with water in the range of 10° to 40°, in particular, in a range of 15° to 30°. Thereby, the adhesive properties of the photocatalyst-containing cell adhesion-inhibiting layer after the energy irradiation can be made higher. The contact angle with water here can be obtained by the method mentioned above.

It is preferable that such a cell adhesion-inhibiting material is contained in the photocatalyst-containing cell adhesion-inhibiting layer in the range of 0.01% by weight to 95% by weight, in particular, in the range of 1% by weight to 10% by weight. Thereby, the region containing the cell adhesion-inhibiting material can be provided as a region with low adhesive properties to a cell.

It is preferable that the above-mentioned cell adhesion-inhibiting material has the interfacial activity. For example, at the time of drying after coating the photocatalyst-containing cell adhesion-inhibiting layer forming coating solution containing the cell adhesion-inhibiting material, the ratio of the uneven distribution on the coating film surface is made higher so that preferable cell adhesion-inhibiting properties can be obtained as a result.

(ii) Others

Moreover, the photocatalyst-containing cell adhesion-inhibiting layer of this embodiment may contain a binder, etc. according to required properties such as coating property at the time of forming a layer, the strength, endurance, etc. when the layer is formed. Moreover, the above-mentioned cell adhesion-inhibiting material may perform the function as the binder.

As such a binder, for example, one having the main skeleton with high bonding energy not to be decomposed by an action of the photocatalyst can be used. Specifically, a polysiloxane having no organic substituent, or having an organic substituent to the extent not to influence the adhesion properties, or the like can be presented. These can be obtained by the hydrolysis and polycondensation of a tetramethoxy silane, a tetraethoxy silane or the like.

In this embodiment, it is preferable that such a binder is contained in the photocatalyst-containing cell adhesion-inhibiting layer by 5% by weight to 95% by weight, more preferably by 40% by weight to 90% by weight, and particularly preferably in a range of 60% by weight to 80% by weight. Thereby, the properties, such as facilitation of the formation of the photocatalyst-containing cell adhesion-inhibiting layer, and provision of the strength to the photocatalyst-containing cell adhesion-inhibiting layer, can be exhibited.

Moreover, in this embodiment, in particular, it is preferable that a cell adhesive material having the adhesive properties to a cell, at least after the energy irradiation, is contained in the photocatalyst-containing cell adhesion-inhibiting layer. Thereby, the photocatalyst-containing cell adhesion-inhibiting layer can be provided with the further preferable adhesive properties to a cell of the cell adhesion portion, which is the energy-irradiated region, that is, the cell culture region. Such cell adhesive material can be used as the binder, and moreover, the cell adhesive material can be used independently from the binder may be used. Furthermore, for example, the cell adhesive material may be a material having preferable adhesive properties to a cell before the irradiation with energy, or a material which obtains preferable adhesive properties to a cell by an action of a photocatalyst upon irradiation with energy. Here, “having adhesive properties to a cell” refers to “adhering preferably to a cell”. In the case the adhesive properties to a cell differ depending on the kind of the cells, it refers to “adhering preferably to the object cells”.

In this embodiment, as long as the cell adhesive material has preferable adhesive properties to a cell at least after irradiation with energy, the adhesion to a cell may be provided preferably by the physical interaction such as the hydrophobic interaction, the electrostatic interaction, the hydrogen bond, and the Van del Waals force, or it may be provided preferably by the biological properties.

In this embodiment, it is preferable that such a cell adhesive material is contained in the photocatalyst-containing cell adhesion-inhibiting layer by 0.01% by weight to 95% by weight, more preferably in a range of 1% by weight to 10% by weight. Thereby, in the photocatalyst-containing cell adhesion-inhibiting layer, the adhesive properties to a cell of the cell adhesion portion, which is the energy-irradiated region, can be improved. Moreover, in the case a material having preferable adhesive properties to a cell before the energy irradiation is used as the cell adhesive material, it is preferable that it is contained to the extent not to inhibit the cell adhesion-inhibiting properties of the above-mentioned cell adhesion-inhibiting material in the region not irradiated with the energy, that is, the region to be the cell adhesion-inhibiting portion.

b. Base Material

Next, the base material used in this embodiment will be explained. The base material used in this embodiment is not particularly limited as long as it is provided with the convex portion mentioned above, and the base material as explained in the above-mentioned first embodiment can be used.

Here, in this embodiment, in the case the base material has the transmission properties to the energy to be irradiated, a light-shielding portion may be formed in the region, provided with the convex portion, of the base material. Thereby, at the time of providing the cell adhesion portion by irradiating the energy to the photocatalyst-containing cell adhesion-inhibiting layer on the cell culture region, by irradiating the energy to the entire surface from the rear surface side of the base material, without the need of using a photomask, the cell adhesion portion can be formed easily. In this embodiment, the light-shielding portion may be formed independently in addition to the convex portion provided on the base material. However, the light-shielding portion may be formed as the convex portion of the base material. Thereby, the cell culture patterning substrate can be formed with good manufacturing efficiency.

Here, since the kind of the base material, the method for forming, the kind, etc. of the light-shielding portion used in this embodiment are same as those explained in the first embodiment. Therefore, detailed explanation is omitted here.

c. Method for Forming a Cell Adhesion Portion

Next, the method for forming a cell adhesion portion will be explained. In this embodiment, the photocatalyst-containing cell adhesion-inhibiting layer on the cell culture region is irradiated with the energy by using a photomask, etc. Thereby, the cell adhesion-inhibiting portion of the energy-irradiated region can be decomposed or denatured so that the cell adhesion portion (cell culture region) having adhesive properties to a cell can be formed. In this case, the cell adhesion portion contains the photocatalyst, decomposed product or denatured product of the cell adhesion-inhibiting material and the like. On the other hand, in the region provided with the convex portion, that is the region not irradiated with the energy, the cell adhesion-inhibiting material still remains so that the cell adhesion-inhibiting portion having no adhesive properties to a cell can be formed.

The energy irradiation (exposure) mentioned in this embodiment is a concept that includes all energy ray irradiation that can decompose or denature the cell adhesion-inhibiting material by the action of a photocatalyst upon irradiation with energy, and is not limited to light irradiation.

Since such energy irradiation method and the like can be the same as that described in the above first embodiment. Therefore, detailed description is omitted.

### (3) THIRD EMBODIMENT

Next, the third embodiment is a cell culture patterning substrate, wherein a photocatalyst-containing layer and a cell adhesion layer are formed on the base material, the photocatalyst-containing layer contains at least a photocatalyst, the cell adhesion layer contains a cell adhesive material which has adhesive properties to a cell, and is to be decomposed or denatured by an action of a photocatalyst upon irradiation with energy, and in the cell adhesion-inhibiting portion, the cell adhesive material has been decomposed or denatured by an action of a photocatalyst upon irradiation with energy.

In this embodiment, since the cell adhesion layer is formed on the photocatalyst containing layer, by irradiating the energy to the region provided with the convex portion, the cell adhesive material in the cell adhesion layer can be decomposed or denatured by the action of the photocatalyst in the adjacent photocatalyst-containing layer. Thereby, the cell adhesion-inhibiting portion, whose adhesive properties to a cell are lowered, can be formed. At the time, for example in the case the cell adhesive material is decomposed by an action of a photocatalyst upon irradiation with energy, the cell adhesion-inhibiting portion contains a small amount of the cell adhesive material, or it contains the decomposed product of the cell adhesive material or the like, or the cell adhesion layer is completely decomposed and removed so as to expose the photocatalyst-containing layer or the like. Moreover, in the case the cell adhesive material is denatured by an action of a photocatalyst upon irradiation with energy, the cell adhesion-inhibiting portion contains the denatured product thereof or the like.

In this embodiment, when the cells are adhered to the cell culture region on the cell culture patterning substrate to obtain the cell culture substrate, by irradiating the energy only to the cell adhesion-inhibiting portion provided with the convex portion, the cells adhered to the cell adhesion-inhibiting portion can be removed by the action of the photocatalyst-containing layer. Thus, there is an advantage that the cells cultured in high-definition pattern can be maintained.

Hereinafter, each configuration of this embodiment will be explained. Since the base material used in this embodiment, and the method for forming the cell adhesion-inhibiting portion in this embodiment are same as those described in the first embodiment, the description thereof is omitted here.

a. Cell Adhesion Layer

First, the cell adhesion layer used in this embodiment will be explained. The cell adhesion layer used in this embodiment is a layer containing at least a cell adhesive material having the adhesive properties to a cell so that a layer commonly used as a layer having the adhesive properties to a cell can be used.

As to the specific cell adhesive material, since the same cell adhesive material used in the photocatalyst-containing cell adhesion layer explained in the first embodiment can be used, the detailed description thereof is omitted here. Moreover, it is preferable that the cell adhesion layer in this embodiment also contains the material having the cell adhesion-inhibiting properties explained for the photocatalyst-containing cell adhesion layer of the first embodiment. Thereby, the adhesive properties to a cell of the cell adhesion-inhibiting portion, which is the region irradiated with the energy, can be made lower.

Moreover, such a cell adhesion layer can be formed by coating a cell adhesion layer forming coating solution, containing the cell adhesive material, by a common coating method or the like. Since it can be same as the method for forming the photocatalyst-containing cell adhesion layer of the first embodiment, the description thereof is omitted here. Moreover, in a case of using relatively expensive cell adhesive material such as protein, adsorption method may be applied for the formation of the cell adhesion layer in some cases.

The film thickness of the cell adhesion layer can be selected optionally according to the kind of the cell culture patterning substrate or the like. It is in general about 0.001 μm to 1.0 μm, in particular, about 0.01 μm to 0.3 μm.

b. Photocatalyst-Containing Layer

Next, the photocatalyst-containing layer used in this embodiment will be explained. The photocatalyst-containing layer used in this embodiment is not particularly limited as long as it is a layer containing at least a photocatalyst. It may be a layer containing only a photocatalyst, or it may be a layer containing other components such as a binder or the like.

The photocatalyst used in this embodiment can be same as those used in the photocatalyst-containing cell adhesion layer in the first embodiment. Also in this embodiment, it is particularly preferable to use a titanium oxide.

The photocatalyst-containing layer consisting of a photocatalyst only is advantageous in costs because the efficiency of decomposing or denaturing the cell adhesive material in the cell adhesion layer is improved to reduce the treatment time. On the other hand, use of the photocatalyst-containing layer comprising a photocatalyst and a binder is advantageous in that the photocatalyst-containing layer can be easily formed.

An example of the method for forming the photocatalyst-containing layer made only of a photocatalyst may be a vacuum film-forming method such as sputtering, CVD or vacuum vapor deposition. The formation of the photocatalyst-containing layer by the vacuum film-forming method makes it possible to render the layer a homogeneous photocatalyst-containing layer made only of a photocatalyst. Thereby, the cell adhesive material can be decomposed or denatured homogeneously. At the same time, since the layer is made only of a photocatalyst, the cell adhesive material can be decomposed or denatured more effectively, as compared with the case of using a binder.

Another example of the method for forming the photocatalyst-containing layer made only of a photocatalyst, is the following method: for example, in the case that the photocatalyst is titanium dioxide, amorphous titania is formed on the base material, and then, calcinating so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is calcinated at 400° C. to 500° C. so as to be denatured to anatase type titania, and calcinated at 600° C. to 700° C. so as to be denatured to rutile type titania.

In the case of using a binder, the binder preferably having a high bonding energy, wherein its main skeleton is not decomposed by photoexcitation of the photocatalyst. Examples of such a binder include the organopolysiloxanes described in the above-mentioned item “Cell adhesion layer”.

In the case of using such an organopolysiloxane as the binder, the photocatalyst-containing layer can be formed by dispersing a photocatalyst, the organopolysiloxane as the binder, and optional additives if needed into a solvent to prepare a coating solution, and coating this coating solution onto the base material. The used solvent is preferably an alcoholic based organic solvent such as ethanol or isopropanol. The coating can be performed by a known coating method such as spin coating, spray coating, dip coating, roll coating and bead coating. When an ultraviolet curable component is contained as the binder, the photocatalyst-containing layer can be formed by curing treatment through the irradiation of ultraviolet rays.

As the binder, an amorphous silica precursor can be used. This amorphous silica precursor is preferably: a silicon compound represented by the general formula SiX₄, wherein X are a halogen, a methoxy group, an ethoxy group, an acetyl group or the like; a silanol which is a hydrolyzate thereof; or a polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst-containing layer can be formed by: dispersing the amorphous silica precursor and particles of a photocatalyst homogeneously into a non-aqueous solvent; hydrolyzing with water content in the air to form a silanol onto a transparent base material; and then subjecting to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content of the photocatalyst in the photocatalyst-containing layer can be set in the range of 5 to 60% by weight, preferably in the range of 20 to 40% by weight. The thickness of the photocatalyst-containing layer is preferably in the range of 0.05 to 10 μm.

Besides the above-mentioned photocatalyst and binder, the surfactant and so on used in the above-mentioned cell adhesion layer can be incorporated into the photocatalyst-containing layer.

In this embodiment, the surface of the photocatalyst-containing layer preferably has low adhesive properties to a cell, for example, due to hydrophilic properties surface or the like. Thus, when the cell adhesion layer is decomposed or the like so as to expose the photocatalyst-containing layer, that region can be a region having low adhesive properties to a cell.

Moreover, in this embodiment, as mentioned above, a light-shielding portion may be formed on the photocatalyst containing layer. Thereby, in the case the energy is irradiated onto the entire surface of the cell adhesion layer, the cell adhesive material contained in the cell adhesion layer, in the region other than the region provided with the light shielding portion, can be decomposed or denatured, without exciting the photocatalyst on the region provided with the light shielding portion. Moreover, in this case, since the photocatalyst in the region provided with the light-shielding portion is not excited, the energy irradiation direction is not particularly limited, and thus it is advantageous.

As the light-shielding portion, since those explained in the first embodiment can be used, the detailed description thereof is omitted here.

### (4) FOURTH EMBODIMENT

Moreover, the fourth embodiment is the cell culture patterning substrate, wherein a photocatalyst-containing layer and a cell adhesion-inhibiting layer are formed on the base material, the photocatalyst-containing layer contains at least a photocatalyst, the cell adhesion-inhibiting layer contains a cell 1adhesion-inhibiting material which has cell adhesion-inhibiting properties of inhibiting adhesion to a cell, and is to be decomposed or denatured by an action of a photocatalyst upon irradiation with energy, and in the cell adhesion portion, the cell adhesion-inhibiting material has been decomposed or denatured by an action of a photocatalyst upon irradiation with energy.

In this embodiment, since the cell adhesion-inhibiting layer is formed on the photocatalyst-containing layer, by irradiating the energy to the cell adhesion-inhibiting layer formed in the region other than the region provided with the convex portion, the photocatalyst contained in the photocatalyst-containing layer is excited so that the cell adhesion-inhibiting material in the cell adhesion-inhibiting layer can be decomposed or denatured. Thus, the cell adhesion portion (cell culture region) can be formed. At the time, the region provided with the convex portion, which is the region not irradiated with the energy where the cell adhesion-inhibiting material still remains, can be the cell adhesion-inhibiting portion.

The phrase “the cell adhesion-inhibiting material is decomposed or denatured” means that the cell adhesion-inhibiting material is not contained, or that the cell adhesion-inhibiting material is contained in a smaller amount than the amount of the cell adhesion-inhibiting material contained in the cell adhesion-inhibiting portion. For example, when the cell adhesion-inhibiting material is decomposed by the action of a photocatalyst upon irradiation with energy, the cell adhesion-inhibiting material is contained in a small amount in the cell adhesion portion, or decomposed products etc. of the cell adhesion-inhibiting material are contained, or the cell adhesion-inhibiting material is completely decomposed to expose the photocatalyst-containing layer. When the cell adhesion-inhibiting material is denatured by the action of a photocatalyst upon irradiation with energy, its denatured products etc. are contained in the cell adhesion portion. In this embodiment, the cell adhesion portion preferably contains the cell adhesive material having adhesive properties to a cell, at least after irradiation with energy. The adhesive properties to a cell of the cell adhesion portion can thereby be increased, and cells can be adhered highly accurately to the cell adhesion portion only.

Hereinafter, the cell adhesion-inhibiting layer used in this embodiment will be explained. The photocatalyst-containing layer same as that described in the above third embodiment can be used in this embodiment. Moreover, the base material and the method for forming the cell adhesion portion used in this embodiment can be the same as that of the above-described second embodiment. Therefore, the explanations are omitted.

a. Cell Adhesion-Inhibiting Layer

The cell adhesion-inhibiting layer used in this embodiment is not particularly limited as long as the layer: is formed on the photocatalyst-containing layer; has cell adhesion-inhibiting properties of inhibiting adhesion to a cell; and contains the cell adhesion-inhibiting material to be decomposed or denatured by an action of a photocatalyst upon irradiation with energy.

In this embodiment, the method for forming the layer is not particularly limited as long as it is capable of forming such layer. For example, the cell adhesion-inhibiting layer can be formed by coating a cell adhesion-inhibiting layer forming coating solution, containing the above-described cell adhesion-inhibiting material, onto the photocatalyst-containing layer by a common coating method. Moreover, the film thickness of the cell adhesion-inhibiting layer can be selected optionally according to the kind, etc. of the cell culture patterning substrate. It is in general about 0.01 μm to 1.0 μm, in particular, about 0.1 μm to 0.3 μm.

The cell adhesion-inhibiting material used in the photocatalyst-containing cell adhesion-inhibiting layer same as that described in the above first embodiment can be used as the specific cell adhesion-inhibiting material used in the cell adhesion-inhibiting layer formed in this embodiment. Therefore, the explanations are omitted. Moreover, the cell adhesion-inhibiting layer in this embodiment preferably contains the material having cell adhesive properties that is explained in the first embodiment for the photocatalyst-containing cell adhesion-inhibiting layer. Thus, the adhesive properties to a cell of the cell adhesion portion (cell culture region), which is the energy-irradiated region, can be improved.

### (5) FIFTH EMBODIMENT

Moreover, the fifth embodiment is the cell culture patterning substrate, wherein a cell adhesion layer is formed, the cell adhesion layer at least contains a cell adhesive material which has adhesive properties to a cell, and is to be decomposed or denatured by an action of a photocatalyst upon irradiation with energy, and in the cell adhesion-inhibiting portion, the cell adhesive material has been decomposed or denatured by an action of a photocatalyst upon irradiation with energy.

In this embodiment, by disposing the cell adhesion layer and the photocatalyst-containing layer facing to each other, and irradiating the energy in a pattern of the cell adhesion-inhibiting portion to be formed, that is the region provided with the convex portion, the cell adhesive material in the cell adhesion layer is decomposed or denatured by the action of the photocatalyst in the photocatalyst-containing layer so that the cell adhesion-inhibiting portion can be formed.

In this embodiment, when the cells are adhered to the cell culture region on the cell culture patterning substrate to obtain the cell culture substrate, by irradiating the energy, using the photocatalyst-containing layer side substrate, to the cell adhesion-inhibiting portion provided with the convex portion, the cells adhered to the cell adhesion-inhibiting portion can be removed by an action of the photocatalyst. Thus, there is an advantage that the cells cultured in a high-definition pattern can be maintained.

Hereinafter, the photocatalyst-containing layer side substrate used in this embodiment, and the method for forming a cell adhesion-inhibiting portion using the photocatalyst-containing layer side substrate will be explained. Since the cell adhesion layer used in this embodiment is same as that used in the third embodiment, the description thereof is omitted here.

a. Photocatalyst-Containing Layer Side Substrate

First, the photocatalyst-containing layer side substrate, comprising a photocatalyst-containing layer containing a photocatalyst, used in this embodiment is described. The photocatalyst-containing layer side substrate used in this embodiment usually comprises a photocatalyst-containing layer containing a photocatalyst, which usually comprises a base body and a photocatalyst-containing layer formed on the base body. This photocatalyst-containing layer side substrate may have, for example, photocatalyst-containing layer side light-shielding portion formed in a pattern form, a primer layer or the like. The following will describe each constituent of the photocatalyst-containing layer side substrate used in this embodiment.

(i) Photocatalyst-Containing Layer

First, the photocatalyst-containing layer used in the photocatalyst-containing layer side substrate is described. The photocatalyst-containing layer used in this embodiment is not particularly limited insofar as the layer is constituted such that the photocatalyst in the photocatalyst-containing layer can cause the decomposition or denaturation of the cell adhesive material in the adjacent cell adhesion layer. The photocatalyst-containing layer may be composed of a photocatalyst and a binder or may be made of a photocatalyst only. The property of the surface thereof may be lyophilic or repellent to liquid.

The photocatalyst-containing layer used in this embodiment may be formed on the whole surface of a base body, or as shown in, for example, FIG. 3, a photocatalyst-containing layer 12 may be formed in a pattern form on a base body 11.

By forming the photocatalyst-containing layer in a pattern accordingly, at the time of irradiating the energy to form the cell adhesion-inhibiting portion, without the need of the pattern irradiation using a photo mask or the like, by the entire surface irradiation, the cell adhesion-inhibiting portion, in which the cell adhesive material contained in the cell adhesion layer is decomposed or denatured, can be formed.

The patterning method for the photocatalyst-containing layer is not particularly limited. It can be carried out by, for example, the photolithography method or the like.

Moreover, since the cell adhesive material only in the portion of the cell adhesion layer actually facing the photocatalyst-containing layer is decomposed or denatured, the energy irradiation direction may be of any direction as long as the energy is irradiated to the portion where the photocatalyst-containing layer and the cell adhesion layer face to each other. Furthermore, there is an advantage that irradiated energy is not particularly limited to parallel ones such as a parallel beam, etc.

Here, since the photocatalyst-containing layer used in this embodiment is same as the photocatalyst-containing layer explained in the third embodiment, the detailed the description thereof is omitted here.

(ii) Base Body

The following will describe the base body used in the photocatalyst-containing layer side substrate. Usually, the photocatalyst-containing layer side substrate comprises at least a base body and a photocatalyst-containing layer formed on the base body. In this case, the material which constitutes the base body to be used is appropriately selected depending on the direction of energy irradiation which will be detailed later, necessity of the resulting pattern-forming body to be transparency, or other factors.

The base body used in this embodiment may be a member having flexibility such as a resin film, or may be a member having no flexibility such as a glass substrate. This is appropriately selected depending on the method of the energy irradiation.

An anchor layer may be formed on the base body in order to improve the adhesion between the surface of the base body and the photocatalyst-containing layer. The anchor layer may be made of, for example, a silane based or titanium based coupling agent.

(iii) Photocatalyst-Containing Layer Side Light-Shielding Portion

As the photocatalyst-containing layer side substrate in this embodiment, a photocatalyst-containing layer side substrate provided with pattern-formed photocatalyst-containing layer side light-shielding portion can be used. When the photocatalyst-containing layer side substrate having photocatalyst-containing layer side light-shielding portion is used in this way, at the time of irradiating energy, it is not necessary to use any photomask or to carry out drawing irradiation with a laser light. Since alignment of the photomask and the photocatalyst-containing layer side substrate is not necessary, process can be made simple. Further, since expensive device for drawing irradiation is also not necessary, it is advantageous in costs.

Such a photocatalyst-containing layer side substrate having photocatalyst-containing layer side light-shielding portion can be classified into the following two embodiments, depending on the position where the photocatalyst-containing layer side light-shielding portion is formed.

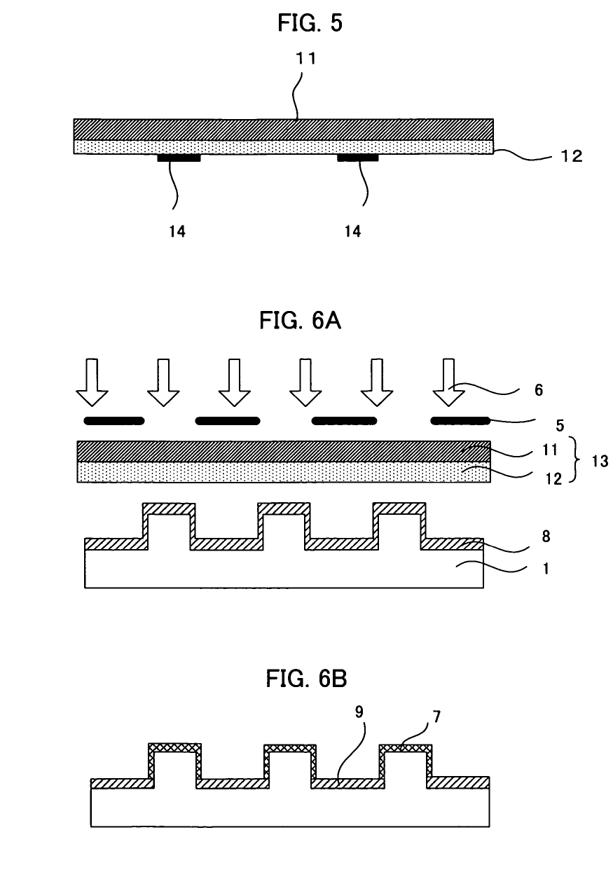

One of them is an embodiment, as shown in FIG. 4 for example, wherein photocatalyst-containing layer side light-shielding portion 14 is formed on a base body 11, and a photocatalyst-containing layer 12 is formed on the photocatalyst-containing layer side light-shielding portion 14 to obtain the photocatalyst-containing layer side substrate. The other example is an embodiment, as shown in FIG. 5 for example, wherein a photocatalyst-containing layer 12 is formed on a base body 11, and photocatalyst-containing layer side light-shielding portion 14 is formed thereon to obtain the photocatalyst-containing layer side substrate.

In any one of these embodiments, since the photocatalyst-containing layer side light-shielding portion is arranged near the region where the photocatalyst-containing layer and the cell adhesion layer are arranged, the effect of energy-scattering in the base body or the like can be made smaller than in the case of using a photomask. Accordingly, irradiation of energy in a pattern can be more precisely attained.

In this embodiment, in the case of the embodiment wherein the photocatalyst-containing layer side light-shielding portion 14 is formed on a photocatalyst-containing layer 12 as shown in FIG. 5, there is an advantage that at the time of arranging the photocatalyst-containing layer and the cell adhesion layer in a predetermined position, the photocatalyst-containing layer side light-shielding portion can be used as a spacer for making the interval constant, by making the film thickness of the photocatalyst-containing layer side light-shielding portion corresponding to the width of the interval between the two layers.

In other words, when the photocatalyst-containing layer and the cell adhesion layer are arranged so as to be facing each other at a predetermined interval, by arranging the photocatalyst-containing layer side light-shielding portion and the cell adhesion layer in close contact to each other, the dimension of the predetermined interval can be made precise. When energy is irradiated in this state, cell adhesion-inhibiting portion can be formed with a good precision since cell adhesive material in the cell adhesion layer, inside the region where the cell adhesion layer and the light-shielding portion are in contact, is not decomposed or denatured.

The method for forming such photocatalyst-containing layer side light-shielding portion is not particularly limited, and may be appropriately selected in accordance with the property of the surface on which the photocatalyst-containing layer side light-shielding portion is to be formed, shielding ability against the required energy, and others. Since the light-shielding portion may be the same as the light-shielding portion provided on the base material that is described in the first embodiment. Thus, the detailed description thereof is omitted herein.

The above has described two cases, wherein the photocatalyst-containing layer side light-shielding portion is formed in between the base body and the photocatalyst-containing layer and wherein it is formed on the surface of the photocatalyst-containing layer. Besides, the photocatalyst-containing layer side light-shielding portion may be formed on the base body surface of the side on which the photocatalyst-containing layer is not formed. In this embodiment, for example, a photomask can be made in close contact to this surface to such a degree that the photomask in removable. Thus, such method can be preferably used for the case that the pattern of the cell adhesion-inhibiting portions is changed for every small lot.

(iv) Primer Layer

The following will describe a primer layer used in the photocatalyst-containing layer side substrate of this embodiment. In this embodiment, when photocatalyst-containing layer side light-shielding portion is formed into a pattern on a base body and a photocatalyst-containing layer is formed thereon so as to prepare a photocatalyst-containing layer side substrate described above, a primer layer may be formed in between the photocatalyst-containing layer side light-shielding portion and the photocatalyst-containing layer.

The effect and function of this primer layer are not necessarily clear, but would be as follows: by forming the primer layer in between the photocatalyst-containing layer side light-shielding portion and the photocatalyst-containing layer, the primer layer is assumed to exhibit a function of preventing the diffusion of impurities from the photocatalyst-containing layer side light-shielding portion and openings present between the photocatalyst-containing layer side light-shielding portions, in particular, residues generated when the photocatalyst-containing layer side light-shielding portion is patterned, or metal or metal ion impurities; the impurities being factors for blocking the decomposition or denaturation of the cell adhesive material by action of the photocatalyst. Accordingly, by forming the primer layer, it is possible to process the decomposition or denaturation of the cell adhesive material with high sensitivity, so as to yield cell adhesion-inhibiting portion which are highly precisely formed.

The primer layer in this embodiment is a layer for preventing the action of the photocatalyst from being affected by the impurities present in not only the photocatalyst-containing layer side light-shielding portion but also in the openings formed between the photocatalyst-containing layer side light-shielding portions. It is therefore preferred to form the primer layer over the entire surface of the photocatalyst-containing layer side light-shielding portion including the openings.

The primer layer in this embodiment is not particularly limited insofar as the primer layer is formed not to bring the photocatalyst-containing layer side light-shielding portion and the photocatalyst-containing layer of the photocatalyst-containing layer side substrate into contact with each other.

A material that forms the primer layer, though not particularly limited, is preferably an inorganic material that is not likely to be decomposed by the action of the photocatalyst. Specifically, amorphous silica can be cited. When such amorphous silica is used, a precursor of the amorphous silica is preferably a silicon compound that is represented by a general formula, SiX₄, wherein X being halogen, methoxy group, ethoxy group, acetyl group or the like; silanol that is a hydrolysate thereof, or polysiloxane having an average molecular weight of 3000 or less.

A film thickness of the primer layer is preferably in the range of 0.001 μm to 1 μm and particularly preferably in the range of 0.001 μm to 0.1 μm.

b. Method for Forming Cell Adhesion-Inhibiting Portion

Hereinafter, the method for forming the cell adhesion-inhibiting portion in this embodiment is described. In this embodiment, for example as shown in FIG. 6, a cell adhesion layer 8 formed on a base material 1, and a photocatalyst-containing layer 12 of a photocatalyst-containing layer side substrate 13, are arranged with a predetermined space and the region provided with the convex portion is irradiated with energy 6 from a predetermined direction, for example, via photomask 5 or the like (FIG. 6A). The cell adhesive material in the region irradiated with energy is thereby decomposed or denatured, thus forming the cell adhesion-inhibiting portion 7, having no cell adhesive properties, in the cell adhesion portion 9 (FIG. 6B). In this case, when the cell adhesive material is decomposed for example by an action of a photocatalyst upon irradiation with energy, the cell adhesion-inhibiting portion contains a small amount of the cell adhesive material or contains decomposed products of the cell adhesive material. Otherwise, the cell adhesion layer is completely decomposed and removed to expose the base material. When the cell adhesive material is denatured by an action of a photocatalyst upon irradiation with energy, its denatured products are contained in the cell adhesion-inhibiting portion.

The above-mentioned wording “arranging” means that the layers are arranged in the state that the action of the photocatalyst can substantially work to the surface of the cell adhesion layer, and include not only the state that the two layers actually contact each other, but also the state that the photocatalyst-containing layer and the cell adhesion layer are arranged at a predetermined interval. The dimension of the interval is preferably 200 μm or less.

In this embodiment, the dimension of the above-mentioned interval is more preferably in the range of 0.2 μm to 10 μm, even more preferably in the range of 1 μm to 5 μm, since the precision of the pattern to be obtained becomes very good and the sensitivity of the photocatalyst becomes high so as to make good efficiency of the decomposition or denaturation of the cell adhesive material in the cell adhesion layer. This range of the interval dimension is particularly effective for the cell adhesion layer which is small in area, wherein the interval dimension can be controlled with a high precision.

Meanwhile, in the case of treating the cell adhesion layer having large area, for example, 300 mm×300 mm or more in size, it is very difficult to make a fine interval as described above in between the photocatalyst-containing layer side substrate and the cell adhesion layer without contacting each other. Accordingly, when the cell adhesion layer has a relatively large area, the interval dimension is preferably in the range of 10 to 100 μm, more preferably in the range of 50 to 75 μm. By setting the interval dimension in the above range, problems will not occur that: deterioration of patterning precision, such as blurring of the pattern; or the sensitivity of the photocatalyst deteriorates so that the efficiency of decomposing or denaturing the cell adhesive material is also deteriorated. Further, there is an advantageous effect that the cell adhesive material is not unevenly decomposed or denatured.

When energy is irradiated onto the cell adhesion layer having a relatively large area as described above, the dimension of the interval, in a unit for positioning the photocatalyst-containing layer side substrate and the cell adhesion layer inside the energy irradiating device, is preferably set in the range of 10 μm to 200 μm, more preferably in the range of 25 μm to 75 μm. The setting of the interval dimension value into this range makes it possible to arrange the photocatalyst-containing layer side substrate and the cell adhesion layer without causing a large deterioration of patterning precision or of sensitivity of the photocatalyst, or bringing the substrate and the layer into contact with each other.

When the photocatalyst-containing layer and the surface of the cell adhesion layer are arranged at a predetermined interval as described above, active oxygen species generated from oxygen and water by action of the photocatalyst can easily be released. In other words, if the interval between the photocatalyst-containing layer and the cell adhesion layer is made narrower than the above-mentioned range, the active oxygen species are not easily released, so as to make the rate for decomposing or denaturing the cell adhesive material unfavorably small. If the two layers are arranged at an interval larger than the above-mentioned range, the generated active oxygen species do not reach the cell adhesion layer easily. In this case also, the rate for decomposing or denaturing the cell adhesive material becomes unfavorably small.

The method for arranging the photocatalyst-containing layer and the cell adhesion layer to make such a very small interval evenly therebetween is, for example, a method of using spacers. The use of the spacers in this way makes it possible to make an even interval. At the same time, the action of the photocatalyst does not work onto the surface of the cell adhesion layer in the regions which the spacers contact. Therefore, when the spacers are rendered to have a pattern similar to that of the cell adhesion portions, the cell adhesive material only inside regions where no spacers are formed can be decomposed or denatured so that highly precise cell adhesion-inhibiting portions can be formed. The use of the spacers also makes it possible that the active oxygen species generated by action of the photocatalyst reach the surface of the cell adhesion layer, without diffusing, at a high concentration. Accordingly, highly precise cell adhesion-inhibiting portion can be effectively formed.

In this embodiment, it is sufficient that such an arrangement state of the photocatalyst-containing layer side substrate is maintained only during the irradiation of energy.

The energy irradiation (exposure) mentioned in this embodiment is a concept that includes all energy ray irradiation that can decompose or denature the cell adhesive material by the action of a photocatalyst upon irradiation with energy, and is not limited to light irradiation.

Here, since the kind, etc. of the energy to be irradiated in this embodiment is same as that explained in the first embodiment, the detailed description thereof is omitted here.

The energy irradiation that is carried out via a photomask in this embodiment, when the above-mentioned base material is transparent, may be carried out from either direction of the base material side or a photocatalyst-containing layer side substrate. On the other hand, when the base material is opaque, it is necessary to irradiate energy from the side of the photocatalyst-containing layer side substrate.

### (6) SIXTH EMBODIMENT

The sixth embodiment is the cell culture patterning substrate, wherein a cell adhesion-inhibiting layer is formed on the base material, the cell adhesion-inhibiting layer contains at least a cell adhesion-inhibiting material which has cell adhesion-inhibiting properties of inhibiting adhesion to a cell, and is to be decomposed or denatured by an action of a photocatalyst upon irradiation with energy, and in the cell adhesion portion, the cell adhesion-inhibiting material has been decomposed or denatured by an action of a photocatalyst upon irradiation with energy.

In this embodiment, the cell adhesion-inhibiting material decomposed or denatured by the action of a photocatalyst upon irradiation with energy is contained in the cell adhesion-inhibiting layer. Therefore, by arranging the cell adhesion-inhibiting layer and the photocatalyst-containing layer so as to be opposite to each other and irradiating with energy in the pattern of the cell adhesion culture region, the cell adhesion-inhibiting material in the cell adhesion-inhibiting layer can be decomposed or denatured by the action of the photocatalyst in the photocatalyst-containing layer to form the cell culture region (cell adhesion portion) having adhesive properties to a cell. Because the cell adhesion-inhibiting material remains in the region not irradiated with energy, this region has no adhesive properties to a cell and can be used as a cell adhesion-inhibiting portion.

The phrase “the cell adhesion-inhibiting material is decomposed or denatured” means that the cell adhesion-inhibiting material is not contained, or that the cell adhesion-inhibiting material is contained in a smaller amount than the amount of the cell adhesion-inhibiting material contained in the cell adhesion-inhibiting portion. For example, when the cell adhesion-inhibiting material is decomposed by the action of a photocatalyst upon irradiation with energy, the cell adhesion-inhibiting material is contained in a small amount in the cell adhesion portion, or decomposed products etc. of the cell adhesion-inhibiting material are contained, or the cell adhesion-inhibiting material is completely decomposed to expose the base material. When the cell adhesion-inhibiting material is denatured by the action of a photocatalyst upon irradiation with energy, its denatured products etc. are contained in the cell adhesion portion. In this embodiment, the cell adhesion portion preferably contains the cell adhesive material having adhesive properties to a cell, at least after irradiation with energy. The adhesive properties to a cell of the cell adhesion portion can thereby be increased, and cells can be adhered highly accurately only to the cell culture region.

The cell adhesion-inhibiting layer used in this embodiment is the same as the cell adhesion-inhibiting layer explained in the above-described fourth embodiment. Also, the photocatalyst-containing layer side substrate, arrangement thereof, energy irradiation method, etc. are the same as that explained in the above-described fifth embodiment. Therefore, detailed description is omitted here.

(Others)

The above-described cell culture patterning substrate may be used for culturing a vascular cell. In this case, a blood vessel can be formed by: culturing the vascular cells which form a blood vessel, in a pattern, on the cell culture region; and adding growth factors which promots vascularization of the vascular cells. Such vascular cells which form a blood vessel refer to vascular endothelial cells, pericytes, smooth muscle cells, vascular endothelial precursor cells and smooth muscle precursor cells derived from organisms, particularly human and animals. Particularly, the vascular endothelial cells etc can be used. Plural kinds of cells can be co-cultured such as co-culture of vascular endothelial cells and pericytes or co-culture of vascular endothelial cells and smooth muscle cells.

Usually, a blood vessel is obtained by forming the vascular cells in an objective pattern on the cell culture region, and then, adding, to a medium, growth factors such as bFGF and VEGF promoting vascularization of the vascular cells. It is estimated that, by stimulation from the growth factors, proliferation of the vascular cells is terminated and differentiated so as to be blood vessels. As the medium for vascularization of the vascular cells adhered in a confluent state to the blood vessel culture region, not only a liquid medium containing the growth factor, but also a gelled medium containing the above-described growth factor or a combination of gelled and liquid mediums containing the growth factor can be used. As the gelled medium, collagen, fibrin gel, Matrigel (trade name) or synthetic peptide hydrogel can be used.

To form blood vessels, it is effective to apply shearing stress in uniaxial direction in the same direction as the line pattern of the cell culture region. The adhered form of the vascular cells can thereby become long and thin spindle-shaped, and the respective vascular cells can adhere to one another in such a state that they seem oriented in the above-mentioned uniaxial direction. To form blood vessels, it is important that the vascular cells are adhered in a confluent state such that the vascular cells are adhered in a thin and long form and the vascular cells are directed to the same direction. The method for applying shear stress in the uniaxial direction includes: a method in which the vascular cells are cultured by placing a culture dish on a shaker or a shaking apparatus; and a method in which the vascular cells are cultured while streaming culture liquid in one direction. To form a blood vessel of 5000 μm or more in width, shearing stress in uniaxial direction is essential.

When a plurality of blood vessels are formed on the cell culture patterning substrate of the present invention, a plurality of cell culture regions are formed as substantially parallel lines. As used herein, the term “parallel” refers not only to completely parallel but also to substantially parallel. That is, the two lines are not crossed in a region. It includes, for example, lines such as zigzag lines that exist without crossing one another. The term “substantially parallel” also refers to portions that are not crossed in a crossed structure such as a net-like structure.

The shape of the cell culture region, on which each blood vessel is formed, is not particularly limited insofar as it is formed in a line form. The shape is selected suitably depending on the shape of an objective blood vessel. Usually, the line width of the cell culture region shall be about 10 μm to 5000 μm, especially 20 μm to 100 μm, particularly 40 μm to 60 μm. A line width of less than 10 μm is not preferable because adhesion of cells is made difficult. A line width of greater than 5000 μm, on the other hand, is not preferable either because almost all vascular cells will be adheres to the cell culture region in a spread state, thus making the cultured vascular cells hardly formable in the form of a blood vessel.

In the present invention, in order to produce a preferable blood vessel, it is particularly preferable that the cell adhesion auxiliary portion is provided in the cell culture region. The cell adhesion auxiliary portion denotes a region having no adhesive properties to a vascular cell, formed as a minute pattern in the cell culture region. The cell adhesion auxiliary portion is formed in a minute pattern to a degree not to inhibit the bonding between the vascular cells in the cell culture region at the time of adhering the vascular cells on the cell culture region. That is, to the degree that the vascular cells can be bound with each other also on the cell adhesion auxiliary portion.

Generally, when vascular cells are adhered to the cell culture region and cultured to form a tissue, the vascular cells are gradually arranged from the outside toward inside of the cell culture region. For forming a tissue, individual vascular cells should be changed morphologically and arranged, and this morphological change of the vascular cell also gradually occurs from the edge part toward center part of the cell culture region. Accordingly, when the width of the cell culture region is large, a tissue may not be formed in the center part of the cell culture region because of insufficient arrangement of the vascular cells, or the vascular cells may fail to adhere to the center part of the cell culture region. Moreover, the morphological change of the vascular cells in the center part of the cell culture region may be insufficient. Therefore, by forming the cell adhesion auxiliary portion described above, the vascular cells can be arranged or morphologically changed from the edge part of the cell adhesion auxiliary portion. Thereby, the vascular cells can be cultured without generating defects or inferior morphological change. Moreover, the cell adhesion auxiliary portion is formed such that vascular cells adjacent to one another via the cell adhesion auxiliary portion are not prevented from being adhered to one another. Thus, the width of the finally cultured vascular cells can be the same as the width of the cell culture region.

The cell adhesion auxiliary portion is formed preferably in a line form in the cell culture region. The shape of the line is not particularly limited and can be in the form of, for example, a straight line, a curved line, a dotted line, a broken line, net-like structure, etc. The line width of the cell adhesion auxiliary portion is preferably in the range of 0.5 μm to 10 μm, more preferably 1 μm to 5 μm. The width larger than the above range is not preferable because the vascular cells adjacent to one another via the cell adhesion auxiliary portion will hardly interact with one another on the cell adhesion auxiliary portion. When the width is smaller than the above range, on the other hand, it is difficult to form by pattern forming techniques in the present invention.

The cell adhesion auxiliary portion may be formed to have a convexoconcave pattern (for example, zigzag etc.) in plane. The term “in plane” refers to the surface of a base material or a surface analogous thereto. The average distance from the edge part of the concave portion to the edge part of the convex portion, of the convexoconcave pattern, may be such a distance that when vascular cells are adhered to the cell culture region, the vascular cells are aligned in the same direction as the line direction of the cell culture region, and the average distance is particularly preferably in the range of 0.5 μm to 30 μm. The average distance from the edge part of the concave portion to the edge part of the convex portion of the convexoconcave pattern is determined by calculating the average of measured distances from the lowermost bottom to the uppermost top of each concavoconvex, within the range of 200 μm of the edge portion of the cell adhesion auxiliary portion. The cell adhesion auxiliary portion can be formed by the same method as the above-described cell adhesion-inhibiting portion.

B. Method for Manufacturing a Cell Culture Patterning Substrate

Next, the method for manufacturing a cell culture patterning substrate of the present invention will be explained. The method for manufacturing a cell culture patterning substrate of the present invention has two embodiments. Hereinafter, each embodiment will be explained independently.

### 1. FIRST EMBODIMENT

First, the first embodiment of the method for manufacturing a cell culture patterning substrate of the present invention will be explained. The first embodiment of the method for manufacturing a cell culture patterning substrate of the present invention is a method for manufacturing a cell culture patterning substrate comprising energy irradiating process of forming a pattern containing a cell adhesion-inhibiting portion and a cell adhesion portion, wherein the cell adhesion-inhibiting portion is a portion whose cell adhesive material contained in a cell adhesion layer has been decomposed or denatured, the cell adhesion portion is a portion having adhesive properties to a cell and an energy is not irradiated thereto, a patterning substrate, comprising: a base material provided with a convex portion; and a cell adhesion layer containing a cell adhesive material which has adhesive properties to a cell and is to be decomposed or denatured by an action of a photocatalyst upon irradiation with energy, is used, a photocatalyst-containing layer side substrate, comprising a base body and a photocatalyst-containing layer containing a photocatalyst, is used, and the pattern is formed by disposing the patterning substrate and the photocatalyst-containing layer side substrate so that the cell adhesion layer and the photocatalyst-containing layer are facing to each other, and irradiating the region provided with the convex portion, with an energy, from a predetermined direction.

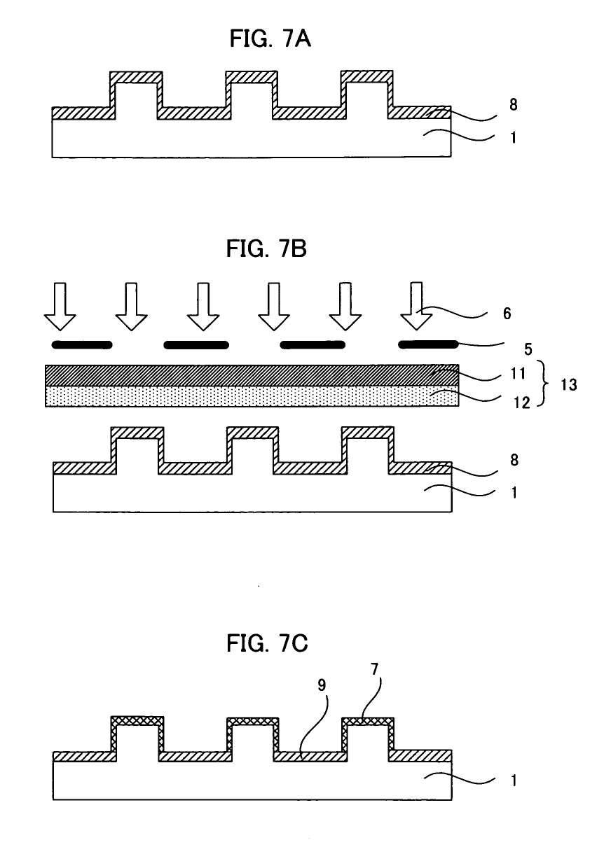

In the energy irradiating process in the method for manufacturing a cell culture patterning substrate of this embodiment, for example as shown in FIG. 7, a patterning substrate comprising a base material 1 provided with a convex portion and a cell adhesive layer 8 formed on the base material 1 is prepared (FIG. 7A). Then, a photocatalyst-containing layer side substrate 13 comprising a photocatalyst-containing layer 12 and a base body 11 is prepared. Disposing the cell adhesive layer 8 of the above-mentioned patterning substrate and the photocatalyst-containing layer 12 facing with each other, an energy 6 is irradiated from a predetermined direction to the region provided with the convex portion by using, for example, a photomask 5 (FIG. 7B). Thus, in this process, a pattern comprising: a cell adhesion-inhibiting portion 7 whose cell adhesive material has been decomposed or denatured by the energy irradiation; and a cell adhesion portion 9 having the adhesive properties to a cell, which is not irradiated with the energy, is formed (FIG. 7C).

In this embodiment, the region, provided with the convex portion, of the manufactured cell culture patterning substrate has the cell adhesion-inhibiting properties. And the region not provided with the convex portion, that is, the cell culture region has the adhesive properties to a cell. Thereby, at the time of adhering the cells on the cell culture region of the cell culture patterning substrate, the adjacent cell culture regions can be partitioned by the convex portion so that bonding of the cells adhered on the adjacent cell culture regions, to each other, can be prevented not only by the height and the width of the above-mentioned convex portion but also by the cell adhesion-inhibiting properties of the region provided with the convex portion. Therefore, a cell culture patterning substrate capable of culturing the cells in a high-definition pattern can be provided.

Since the base material provided with the convex portion and the cell adhesive layer in the patterning substrate used for the energy irradiating process of this embodiment, the photocatalyst-containing layer side substrate, arrangement of the photocatalyst-containing layer side substrate and the cell adhesive layer, the energy to be irradiated, etc. can be same as those explained in the fifth embodiment of the above-mentioned “A. Cell culture patterning substrate”. Therefore, detailed explanation is omitted here.

Moreover, the method for manufacturing a cell culture patterning substrate of this embodiment may optionally include necessary processes in addition to the above-mentioned energy irradiating process. Moreover, in this embodiment, at the time of providing a cell culture substrate by adhering the cells on the cell culture region on the cell culture patterning substrate, by irradiating the energy to the cell adhesion-inhibiting portion provided with the convex portion using the photocatalyst-containing layer side substrate, the cells adhered on the cell adhesion-inhibiting portion can be removed. Thus, there is an advantage that the cells cultured in high-definition pattern can be maintained.

### 2. SECOND EMBODIMENT

Next, the second embodiment of the method for manufacturing a cell culture patterning substrate of the present invention will be explained. The method for manufacturing a cell culture patterning substrate of this embodiment is a method for manufacturing a cell culture patterning substrate comprising energy irradiating process of forming a pattern containing a cell adhesion portion and a cell adhesion-inhibiting portion, wherein the cell adhesion portion is a portion whose cell adhesion-inhibiting material contained in a cell adhesion-inhibiting layer has been decomposed or denatured to have adhesive properties to a cell, the cell adhesion-inhibiting portion is a portion having cell adhesion-inhibiting properties of inhibiting adhesion to a cell and an energy is not irradiated thereto, a patterning substrate, comprising: a base material provided with a convex portion; and a cell adhesion-inhibiting layer containing a cell adhesion-inhibiting material which has cell adhesion-inhibiting properties of inhibiting adhesion to a cell and is to be decomposed or denatured by an action of a photocatalyst upon irradiation with energy, is used, a photocatalyst-containing layer side substrate, comprising a base body and a photocatalyst-containing layer containing a photocatalyst, is used, and the pattern is formed by disposing the patterning substrate and the photocatalyst-containing layer side substrate so that the cell adhesion layer and the photocatalyst-containing layer are facing to each other, and irradiating the region other than the region provided with the convex portion, with an energy, from a predetermined direction.

In this embodiment, since the cell adhesion-inhibiting material still remains in the region provided with the convex portion of the manufactured cell culture patterning substrate, it has the cell adhesion-inhibiting properties. And in the energy-irradiated region not provided with the convex portion, that is, the cell culture region, since the cell adhesion-inhibiting material has been decomposed or denatured, it has the adhesive properties to a cell. Thereby, at the time of adhering the cells on the cell culture region of the cell culture patterning substrate, the adjacent cell culture regions are partitioned by the convex portion so that bonding of the cells adhered on the adjacent cell culture regions, to each other, can be prevented not only by the height and the width of the convex portion but also by the cell adhesion-inhibiting properties of the region provided with the convex portion. Therefore, a cell culture patterning substrate capable of culturing the cells in a high-definition pattern can be provided.

Since the photocatalyst-containing layer side substrate, arrangement of the photocatalyst-containing layer side substrate and the cell adhesive layer, the energy to be irradiated, etc. used in the energy irradiating process of this embodiment can be same as those explained in the fifth embodiment of the above-mentioned “A. Cell culture patterning substrate”. The base material and the cell adhesion-inhibiting layer used in the patterning substrate can be same as those explained in the fourth embodiment of the above-mentioned “A. Cell culture patterning substrate”. Therefore, detailed explanation is omitted here. Moreover, the method for manufacturing a cell culture patterning substrate of this embodiment may optionally include necessary processes in addition to the above-mentioned energy irradiating process.

The present invention is not limited to the above mentioned embodiments. The above mentioned embodiments are merely examples, and any one having the substantially same configuration and the same effects, or equivalent thereof, as the technological idea disclosed in the claims of the present invention is included in the technological scope of the present invention.

### EXAMPLES

Hereafter, with reference to the examples, the present invention will be explained further specifically.

### Example 1

Production of a Base Material Provided with the Convex Portion

Resin line-shaped convex portions of 300 μm width and 25 μm height were formed on a slide glass by a screen printing method so that the pitch of the convex portions is 500 μm.

(Formation of a Photocatalyst-Containing Cell Adhesion-Inhibiting Layer)

3 g of an isopropyl alcohol, 0.4 g of an organosilane TSL8114 (GE Toshiba Silicones), 0.4 g of a fluoroalkyl silane TSL8223 (GE Toshiba Silicones) as the cell adhesion-inhibiting material, and 1.5 g of a photocatalyst inorganic coating agent ST-KO1 (Ishihara Sangyo Kaisha, Ltd.) were mixed and heated at 100° C for 20 minutes while agitating.

By coating the solution onto the above-mentioned slide glass provided with the convex portions by the spin coating method and drying the substrate at a 150° C., temperature for 10 minutes for promoting hydrolysis and polycondensation reaction, a photocatalyst-containing cell adhesion-inhibiting layer, wherein the photocatalyst is strongly tightened in the organopolysiloxane, was formed on the substrate.

The average height difference of the region interposed between the adjacent convex portions and the convex portions after formation of the above-mentioned coating layer was 24.6 μm.

(Patterning of the Photocatalyst-Containing Cell Adhesion-Inhibiting Layer)

A mask provided with line-shaped light-shielding portions of 300 μm width and 500 μm pitch was disposed on a glass provided with the above-mentioned photocatalyst-containing cell adhesion-inhibiting layer such that the convex portions and the light-shielding portions are facing with each other, and ultraviolet ray was irradiated from an exposing machine by 5.5 Jcm⁻². Thereby, the cell adhesion-inhibiting material of the exposed portion was decomposed and removed partially so as to be changed to a region having the cell adhesive properties.

(Dissemination of Cells and Formation of Tissue)

For details of the culturing experiment procedure of the cells derived from various kinds of tissues, refer to, for example, “Soshikibaiyou no Gijutu, third edition, basic edition”, compiled by Japan Tissue Culture Association, Asakura Publishing Co., Ltd. In the present application, the substrate was evaluated by using a rat hepatic parenchymal cell.

The liver taken out from a rat placed in a Petri dish was cut finely to a 5 mm size with a scalpel. After adding 20 ml of a DMEM culture medium and suspending lightly with a pipette, it was filtrated with a cell filtrating device. The centrifugal treatment was applied to the obtained coarse dispersion cell floating solution for 90 seconds by 500 to 600 rpm, and the supernatant on the top was suctioned and removed. To the residual cells, additionally a DMEM culture medium was added and the centrifugal treatment was applied again. By repeating this operation for three times, a substantially homogeneous hepatic parenchymal cell was obtained. By adding 20 ml of a DMEM culture medium to the obtained hepatic parenchymal cells and suspending, a hepaticparenchymal cell suspension was prepared.

Next, 900 ml of distilled water was added to 14.12 g of a Waymouth MBZ752/1 culture medium (containing L-glutamine, not containing NaHCO₃) (manufactured by GIBUCO Corp.). 2.24 g of NaHCO₃, 10 ml of ANHOTERISINE B solution (ICN) and 10 ml of penicillin streptomycin solution (manufactured by GIBUCO Corp.). were added thereto and agitated. After preparing the same to pH 7.4, with the total amount provided as 1,000 ml, it was filtrated with a 0.22 μm membrane filter for reducing germs so as to provide a Waymouth MB752/1 culture medium solution.

After suspending the hepatic parenchymal cell suspension produced preliminarily in the produced Waymouth MB752/1 culture medium solution, dissemination was carried out on the above-mentioned cell culture patterning substrate comprising the cell adhesion portion and the cell adhesion-inhibiting portion. By placing still the substrate in an incubator at 37° C., provided with 5% CO₂ for 24 hours, the hepatic parenchymal cell was adhered on the entire surface of the substrate. After removing the non-adhered cells and the dead cells by washing the substrate with PBS twice, it was replaced with a new culture medium solution.

According to the observation of the cells with an optical microscope after continuing the cell culture for 48 hours while replacing the culture medium solution, it was confirmed that the cells were adhered along the cell adhesion portion interposed between the convex portions, that is, the cell culture region of the cell culture patterning substrate. Moreover, it was confirmed that no adhesion of the cells with each other between the above-mentioned cell adhesion regions (on the convex portion).

### Example 2

(Production of a Base Material Provided with the Convex Portion)

Resin line-shaped convex portions of 200 μm width and 30 μm height were formed on a transparent polystyrene plate, by the same method as Example 1, so that the pitch of the convex portions is 400 μm.

(Formation of a Photocatalyst-Containing Cell Adhesion-Inhibiting Layer)

3 g of an isopropyl alcohol, 0.4 g of an organosilane TSL8114 (GE Toshiba Silicones), 0.4 g of a aminopropyltriethoxysilane as the cell adhesive material, and 1.5 g of a photocatalyst inorganic coating agent ST-K01 (Ishihara Sangyo Kaisha, Ltd.) were mixed and heated at 100° C. for 20 minutes while agitating.

By coating the solution onto the above-mentioned polystyrene plate provided with the convex portions by the spin coating method and drying the substrate at a 150° C. temperature for 10 minutes for promoting hydrolysis and polycondensation reaction, a photocatalyst-containing cell adhesion layer, wherein the photocatalyst is strongly tightened in the organopolysiloxane, was formed on the substrate.

The average height difference of the region interposed between the adjacent convex portions and the convex portions after formation of the above-mentioned coating layer was 29.5 μm.

(Patterning of the Photocatalyst-Containing Cell Adhesion Layer)

A mask provided with line-shaped light-shielding portions of 200 μm width and 400 μm pitch was disposed on the polystyrene plate provided with the above-mentioned photocatalyst-containing cell adhesion layer such that the region interposed between the convex portions and the light-shielding portions are facing with each other, and ultraviolet ray was irradiated from an exposing machine by 12 Jcm⁻². Thereby, the cell adhesive material of the exposed portion was decomposed and removed almost entirely so as to be changed to a region of extremely highly hydrophilic having the cell adhesion-inhibiting properties.

(Dissemination of Cells and Formation of Tissue)

The cells were disseminated in the same manner as Example 1. As in Example 1, it was confirmed that the cells were adhered along the cell adhesion portion interposed between the convex portions, that is, the cell culture region on the cell culture patterning substrate. Moreover, it was confirmed that no adhesion of the cells with each other between the above-mentioned cell adhesion regions (on the convex portion).

### Example 3

(Production of a Base Material Provided with the Convex Portion)

The base material same as that used in Example 1 was used.

(Formation of a Photocatalyst-Containing Layer)

3 g of an isopropyl alcohol, 0.4 g of an organosilane TSL8114 (GE Toshiba Silicones), and 1.5 g of a photocatalyst inorganic coating agent ST-K01 (Ishihara Sangyo Kaisha, Ltd.) were mixed and heated at 100° C. for 20 minutes while agitating.

By coating the solution onto the above-mentioned slide glass provided with the convex portions by the spin coating method and drying the substrate at a 150° C. temperature for 10 minutes for promoting hydrolysis and polycondensation reaction, a photocatalyst-containing layer, wherein the photocatalyst is strongly tightened in the organopolysiloxane, was formed on the substrate.

The average height difference of the region interposed between the adjacent convex portions and the convex portions after formation of the above-mentioned photocatalyst-containing layer was 24.7 μm.

(Formation of the Cell Adhesion Layer)

0.2 mg of fibronectin F-4759 (Sigma) and 200 ml of pure water were mixed. The aqueous solution was dripped onto the photocatalyst-containing layer of the slide glass in the ratio of 300 μm per 1 cm² of substrate surface, and this was placed still for 24 hours under 4° C. Further, the substrate was washed with PBS for twice, and the slide glass, comprising the photocatalyst-containing layer and cell adhesion layer, on the base material provided with the convex portion was obtained.

(Patterning of the Cell Adhesion Layer)

A mask provided with line-shaped light-shielding portions of 200 μm width and 500 μm pitch was disposed such that the region interposed between the convex portions on the slide glass and the light-shielding portions are facing with each other, and ultraviolet ray was irradiated from an exposing machine by 12 Jcm⁻². Thereby, in the exposed convex portion surface, the fibronectin as the cell adhesive material was decomposed and removed almost entirely so as to be changed to a region of extremely highly hydrophilic having the cell adhesion-inhibiting properties.

(Dissemination of Cells and Formation of Tissue)

As in Example 1, the cell adhesion experiment was carried out. As in Example 1, it was confirmed that the cells were adhered along the cell adhesion portion interposed between the convex portions, that is, the cell culture region on the cell culture patterning substrate. Moreover, it was confirmed that no adhesion of the cells with each other between the above-mentioned cell adhesion regions (on the convex portion).

### Example 4

(Production of a Base Material Provided with the Convex Portion)

The base material same as that used in Example 1 was used.

(Formation of the Cell Adhesion Layer)

0.2 mg of fibronectin F-4759 (Sigma) and 200 ml of pure water were mixed. The aqueous solution was dripped onto the slide glass at the ratio of 300 μm per 1 cm² of substrate surface, and this was placed still for 24 hours under 4° C. Further, the substrate was washed with PBS for twice, and the slide glass, comprising the cell adhesion layer, on the base material provided with the convex portion was obtained.

<Production of the Photocatalyst-Containing Layer Side Substrate>

(Production of the Photocatalyst-Containing Layer Forming Coating Solution)

3 g of an isopropyl alcohol, 0.4 g of an organosilane TSL8114 (GE Toshiba Silicones), and 1.5 g of a photocatalyst inorganic coating agent ST-K01 (Ishihara Sangyo Kaisha, Ltd.) were mixed and heated at 100° C. for 20 minutes while agitating.

(Formation of a Photocatalyst-Containing Layer)

According to a common procedure for producing a chromium mask, a photomask provided with stripe-shaped photocatalyst-containing layer side light-shielding portion, comprising 200 μm light-shielding portions and 300 μm opening portions (500 μm pitch), was produced on a quartz glass. By coating the photocatalyst-containing layer forming coating solution onto this photomask surface by the spin coating method and drying the substrate at a 150° C. temperature for 10 minutes for promoting hydrolysis and polycondensation reaction, a photocatalyst-containing layer of 0.2 μm thickness, wherein the photocatalyst is strongly tightened in the organopolysiloxane, was formed on the photomask, thus obtaining the photocatalyst-containing layer side substrate.

(Patterning of the Cell Adhesion Layer)

The photocatalyst-containing layer side substrate was disposed such that the region interposed between the convex portions on the slide glass and the light-shielding portions are facing with each other, and ultraviolet ray was irradiated from an exposing machine by 12 Jcm⁻²Thereby, in the exposed convex portion surface, the fibronectin as the cell adhesive material was decomposed and removed almost entirely so as to be changed to a region of extremely highly hydrophilic having the cell adhesion-inhibiting properties.

(Dissemination of Cells and Formation of Tissue)

As in Example 1, the cell adhesion experiment was carried out. As in Example 1, it was confirmed that the cells were adhered along the cell adhesion portion interposed between the convex portions, that is, the cell culture region on the cell culture patterning substrate. Moreover, it was confirmed that no adhesion of the cells with each other between the above-mentioned cell adhesion regions (on the convex portion).

### Example 5

(Production of a Base Material Provided with the Convex Portion)

Resin line-shaped convex portions of 300 μm width and 25 μm height were formed on a slide glass by a screen printing method so as the pitch of the convex portions is 700 μm.

(Formation of a Cell Adhesion-Inhibiting Layer)

A cell adhesion-inhibiting layer was formed by: diluting silane coupling agent XC98-B2472 (GE Toshiba Silicones) with isopropyl alcohol by 10 times; adding thereto 1,3-butane diol so as to have 10% concentration and agitating; preparing a coating agent for forming a cell adhesion-inhibiting layer; UV-cleaning the slide glass provided with the above-mentioned line-shaped convex portions for 120 seconds; coating the above-mentioned coating agent immediately thereafter by the spin coating method; and drying at 150° C. for 15 minutes,.

(Production of the Photocatalyst-Containing Layer Side Substrate)

A photomask having 300 μm width line-shaped light-shielding portions and 400 μm width line-shaped opening portions formed alternately was produced. At the time, in the 400 μm width line-shaped opening portion, line-shaped light-shielding portions of 40 μm pitch and 5 μm width were formed along the line direction of the opening portion as a portion corresponding to the cell adhesion auxiliary portion. By forming a photocatalyst-containing layer on the photomask in the same manner as in the example 4, a transparent photocatalyst-containing layer side substrate was provided.

(Patterning of the Cell Adhesion-Inhibiting Layer)

The photocatalyst-containing layer of the photocatalyst-containing layer side substrate having a pattern corresponding to the above-mentioned cell adhesion auxiliary portion, and the above-mentioned cell adhesion-inhibiting layer were disposed so as to be facing with each other. At the time, positioning was carried out so as to have the above-mentioned line-shaped light-shielding portion provided on the line-shaped convex portion of the above-mentioned base material. Subsequently, ultraviolet ray was irradiated by 6 J/cm² from the photocatalyst-containing layer side substrate side. Thereby, the cell adhesion-inhibiting material in the region interposed between the adjacent convex portions, among the cell adhesion-inhibiting layer formed on the base material, was removed in a pattern. Thus, a cell adhesion portion having a cell adhesion auxiliary portion was formed.

(Dissemination of Cells and Formation of Tissue)

With a cell culture patterning substrate provided with the cell adhesion portion placed on a culture dish containing culture solution (5% bovine fetal serum containing MEM culture medium), a bovine vascular endothelial cell was disseminated so as to have a 6×10⁵ pieces/ml concentration. The culture dish was placed on a seesaw shaker for culturing for 24 hours so that the endothelial cells were adhered on the cell adhesion portion having the cell adhesion auxiliary portion. The shaker was adjusted so as to be operated slowly like a seesaw for generating the culture medium flow in the same direction as the line pattern of the cell culture patterning substrate. According to the microscope observation, it was confirmed that the adhered vascular endothelial cells have a shape oriented along the line direction.

1. A cell culture patterning substrate comprising:
a base material provided with a convex portion; and a cell culture region, which is a region for culturing a cell, formed on a surface of the base material,
wherein the cell culture region is partitioned with the convex portion of the base material provided with the convex portion.

2. The cell culture patterning substrate according to claim 1, wherein a region provided with the convex portion is a cell adhesion-inhibiting portion having adhesion-inhibiting properties to a cell, and
the cell culture region is a cell adhesion portion having adhesive properties to a cell.

3. The cell culture patterning substrate according to claim 2, wherein a photocatalyst-containing cell adhesion layer is formed on the base material,
the photocatalyst-containing cell adhesion layer contains at least: a photocatalyst; and a cell adhesive material which has adhesive properties to a cell, and is to be decomposed or denatured by an action of a photocatalyst upon irradiation with energy, and
in the cell adhesion-inhibiting portion, the cell adhesive material has been decomposed or denatured by an action of a photocatalyst upon irradiation with energy.

4. The cell culture patterning substrate according to claim 2, wherein a photocatalyst-containing cell adhesion-inhibiting layer is formed on the base material,
the photocatalyst-containing cell adhesion-inhibiting layer contains at least: a photocatalyst; and a cell adhesion-inhibiting material which has cell adhesion-inhibiting properties of inhibiting adhesion to a cell, and is to be decomposed or denatured by an action of a photocatalyst upon irradiation with energy, and
in the cell adhesion portion, the cell adhesion-inhibiting material has been decomposed or denatured by an action of a photocatalyst upon irradiation with energy.

5. The cell culture patterning substrate according to claim 2, wherein a photocatalyst-containing layer and a cell adhesion layer are formed on the base material,
the photocatalyst-containing layer contains at least a photocatalyst,
the cell adhesion layer contains a cell adhesive material which has adhesive properties to a cell, and is to be decomposed or denatured by an action of a photocatalyst upon irradiation with energy, and
in the cell adhesion-inhibiting portion, the cell adhesive material has been decomposed or denatured by an action of a photocatalyst upon irradiation with energy.

6. The cell culture patterning substrate according to claim 2, wherein a photocatalyst-containing layer and a cell adhesion-inhibiting layer are formed on the base material,
the photocatalyst-containing layer contains at least a photocatalyst,
the cell adhesion-inhibiting layer contains a cell adhesion-inhibiting material which has cell adhesion-inhibiting properties of inhibiting adhesion to a cell, and is to be decomposed or denatured by an action of a photocatalyst upon irradiation with energy, and
in the cell adhesion portion, the cell adhesion-inhibiting material has been decomposed or denatured by an action of a photocatalyst upon irradiation with energy.

7. The cell culture patterning substrate according to claim 2, wherein a cell adhesion layer is formed on the base material,
the cell adhesion layer contains a cell adhesive material which has adhesive properties to a cell, and is to be decomposed or denatured by an action of a photocatalyst upon irradiation with energy, and
in the cell adhesion-inhibiting portion, the cell adhesive material has been decomposed or denatured by an action of a photocatalyst upon irradiation with energy.

8. The cell culture patterning substrate according to claim 2, wherein a cell adhesion-inhibiting layer is formed on the base material,
the cell adhesion-inhibiting layer contains a cell adhesion-inhibiting material which has cell adhesion-inhibiting properties of inhibiting adhesion to a cell, and is to be decomposed or denatured by an action of a photocatalyst upon irradiation with energy, and
in the cell adhesion portion, the cell adhesion-inhibiting material has been decomposed or denatured by an action of a photocatalyst upon irradiation with energy.

9. A method for manufacturing a cell culture patterning substrate comprising energy irradiating process of forming a pattern containing a cell adhesion-inhibiting portion and a cell adhesion portion,
wherein the cell adhesion-inhibiting portion is a portion whose cell adhesive material contained in a cell adhesion layer has been decomposed or denatured,
the cell adhesion portion is a portion having adhesive properties to a cell and an energy is not irradiated thereto,
a patterning substrate, comprising: a base material provided with a convex portion; and a cell adhesion layer containing a cell adhesive material which has adhesive properties to a cell and is to be decomposed or denatured by an action of a photocatalyst upon irradiation with energy, is used,
a photocatalyst-containing layer side substrate, comprising a base body and a photocatalyst-containing layer containing a photocatalyst, is used, and
the pattern is formed by disposing the patterning substrate and the photocatalyst-containing layer side substrate so that the cell adhesion layer and the photocatalyst-containing layer are facing to each other, and irradiating the region provided with the convex portion, with an energy, from a predetermined direction.

10. A method for manufacturing a cell culture patterning substrate comprising energy irradiating process of forming a pattern containing a cell adhesion portion and a cell adhesion-inhibiting portion,
wherein the cell adhesion portion is a portion whose cell adhesion-inhibiting material contained in a cell adhesion-inhibiting layer has been decomposed or denatured to have adhesive properties to a cell,
the cell adhesion-inhibiting portion is a portion having cell adhesion-inhibiting properties of inhibiting adhesion to a cell and an energy is not irradiated thereto,
a patterning substrate, comprising: a base material provided with a convex portion; and a cell adhesion-inhibiting layer containing a cell adhesion-inhibiting material which has cell adhesion-inhibiting properties of inhibiting adhesion to a cell and is to be decomposed or denatured by an action of a photocatalyst upon irradiation with energy, is used,
a photocatalyst-containing layer side substrate, comprising a base body and a photocatalyst-containing layer containing a photocatalyst, is used, and
the pattern is formed by disposing the patterning substrate and the photocatalyst-containing layer side substrate so that the cell adhesion layer and the photocatalyst-containing layer are facing to each other, and irradiating the region other than the region provided with the convex portion, with an energy, from a predetermined direction.
